(19) United States
(12) Patent Application Publication
(10) Pub. No.: US 2018/0334429 A1
(43) Pub. Date: Nov. 22, 2018

(54) PROCESS FOR PREPARATION OF APREMILAST AND ITS INTERMEDIATES

(71) Applicant: DR. REDDY'S LABORATORIES LIMITED, Hyderabad (IN)

(72) Inventors: Sree Naga Venkata Lakshmi Vara Prasad VAKAMUDI, Hyderabad (IN); Syam Kumar Unniaran KUNHIMON, Thrissur (IN); Soma Rani SARKAR, Hyderabad (IN); Babu IRENI, Hyderabad (IN); Amarnath Reddy LEKKALA, Hyderabad (IN); Gangadhara Chary RAPAKA, Hyderabad (IN); Srinivasa Rao MADARAPU, Hyderabad (IN); Ramesh Kumar NADGOUD, Hyderabad (IN); Sridhar CHAGANTI, Hyderabad (IN); Venkateswarlu MUVVA, Hyderabad (IN); Anna FRYSZKOWSKA, Cambridge (GB); Martin FOX, Cambridge (GB); Tamara Fanjul SOLARES, Cambridge (GB); Vijay Kumar SHANGAPU, Hyderabad (IN); Nilesh HASTAK, Hyderabad (IN); Sunitha VYALA, Hyderabad (IN); Srininvasulu RANGINENI, Hyderabad (IN)

(21) Appl. No.: 15/580,625

(22) PCT Filed: Jun. 8, 2016
(86) PCT No.: PCT/IB2016/053351

§ 371 Date: Jun. 27, 2018

(30) Foreign Application Priority Data
Jun. 9, 2015 (IN) 2879/CHE/2015
Aug. 13, 2015 (IN) 4212/CHE/2015
Sep. 9, 2015 (IN) 4780/CHE/2015
Sep. 9, 2015 (IN) 4787/CHE/2015

(51) Int. Cl. C07C 315/04; C07C 315/06; C07C 317/28

(52) U.S. Cl. CPC C07C 315/04; C07C 317/28; C07C 315/06

(57) ABSTRACT

Present application relates to the process for the preparation of 1-(3-Ethoxy-4-methoxy-phenyl)-2-methanesulf-onyl-ethylamine of the formula (II), its resolution and its use in preparation of Apremilast of formula (I), process for the preparation of crystalline form B of apremilast, process for preparation of amorphous form of apremilast and the crystalline form of (S)-1-(3-Eth-oxy-4-methoxy-phenyl)-2-methanesulfonyl-ethylamine of the formula (Va).

### FIELD OF THE APPLICATION

Present application relates to the process for the preparation of 1-(3-Ethoxy-4-methoxy-phenyl)-2-methanesulfonyl-ethylamine of the formula (II) and its use in preparation of apremilast. Another aspect of the present application provides process for resolution of 1-(3-Ethoxy-4-methoxy-phenyl)-2-methanesulfonyl-ethylamine of the formula (II) and its use in preparation of apremilast. Another aspect of the present application provides process for preparation of (S)-1-(3-Ethoxy-4-methoxy-phenyl)-2-methanesulfonyl-ethylamine of the formula (Va) and its crystalline form. Another aspect of the present application provides a process for preparation of racemic 1-(3-ethoxy-4-methoxy-phenyl)-2-methanesulfonyl-ethylamine of formula (II) from (R)-1-(3-ethoxy-4-methoxy-phenyl)-2-methanesulfonyl-ethylamine of formula (Vb). Still another aspect of the present application provides process for the preparation of crystalline form B of apremilast and also process for preparation of amorphous form of apremilast.

### BACKGROUND

Apremilast is a PDE4 inhibitor and acts as an anti-inflammatory agent for the treatment of a variety of conditions, including asthma, chronic obstructive pulmonary disease, psoriasis and other allergic, autoimmune and rheumatologic conditions and is represented by structure of formula (I).

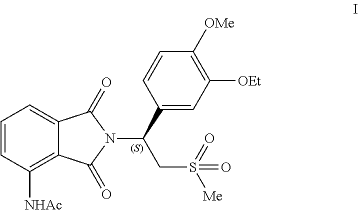

1-(3-Ethoxy-4-methoxy-phenyl)-2-methanesulfonyl-ethylamine of formula (II) and its (S)-isomer of formula (Va) are the key intermediates useful in the preparation of apremilast.

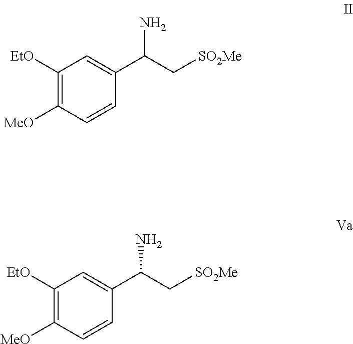 [SMILES: CCOc1cc(C(N)CS(C)(=O)=O)ccc1OC ; CCOc1cc([C@H](N)CS(C)(=O)=O)ccc1OC]

Synthetic routes to apremilast often rely on classical resolution of racemic amino sulfone (II) to obtain required (S)-enantiomer (Va), which results in significant waste stream of the unwanted (R)-enantiomer of formula (Vb), herein after referred as (R)-aminosulfone.

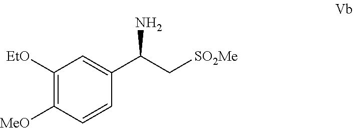 [SMILES: CCOc1cc([C@@H](N)CS(C)(=O)=O)ccc1OC]

Processes for the preparation of apremilast and its intermediates have been disclosed in U.S. Pat. No. 6,020,358B2, U.S. Pat. No. 7,427,638B2, U.S. Pat. No. 8,242,310B2, US20130217918A1 and US20130217919A1.

In view of the importance of PDE4 inhibitors, cost-effective and novel methods of making such drugs and their intermediates are always of interest. The present invention provides a cost and yield-improving process to prepare Apremilast or its intermediate and also to recycle the (R)-amino sulfone (Vb) to racemic aminosulfone (II).

### BRIEF DESCRIPTION OF THE DRAWINGS

FIG. 1 is an illustration of a PXRD pattern of chiral aminosulfone of formula (Va), obtained in the present invention.

FIG. 2 is an illustration of a PXRD pattern of amorphous form of apremilast obtained by the example 20.

### SUMMARY OF THE APPLICATION

The present application provides novel synthetic processes for obtaining Apremilast of formula (I) and its related intermediates.

In first embodiment, the present application provides a process for preparation of aminosulfone of formula (II) or its stereo isomers and their pharmaceutically acceptable salts

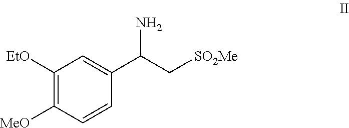 [SMILES: CCOc1cc(C(N)CS(C)(=O)=O)ccc1OC]

which comprises:

(a) reacting benzonitrile of formula (III) with dimethyl sulfone in the presence of a base in a suitable solvent to provide a compound of formula (IVa), followed by its conversion to provide enamine of formula (IV);

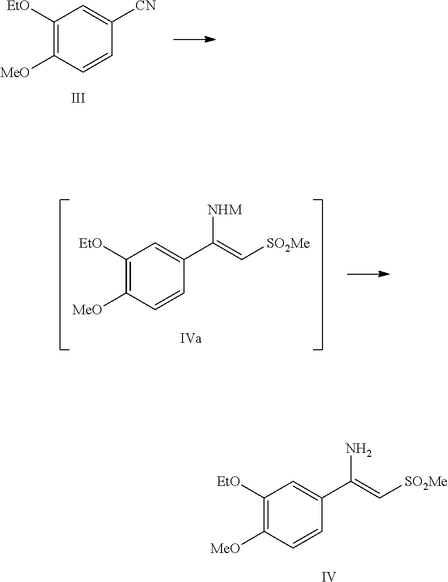

Wherein M=Na, K

(b) reducing enamine of formula (IV) in presence of a suitable solvent to provide aminosulfone of formula (II);

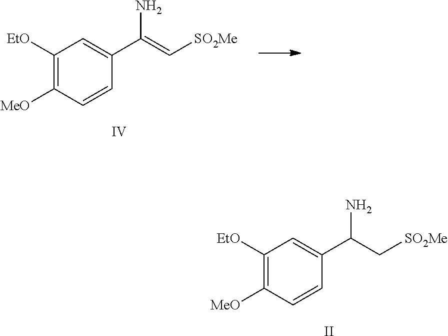

(c) optionally purifying amino sulfone of formula (II).

In second embodiment, the present application provides a process for the preparation of formula (Va) or its stereoisomers thereof

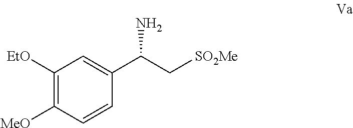 [SMILES: CCOc1cc([C@H](N)CS(C)(=O)=O)ccc1OC]

which comprises:

(a) contacting racemic 1-(3-ethoxy-4-methoxy-phenyl)-2-methanesulfonyl-ethylamine of formula (II) with a chiral acid in presence of a suitable solvent to form a chiral acid salt of 1-(3-ethoxy-4-methoxy-phenyl)-2-methanesulfonyl-ethylamine of formula (V);

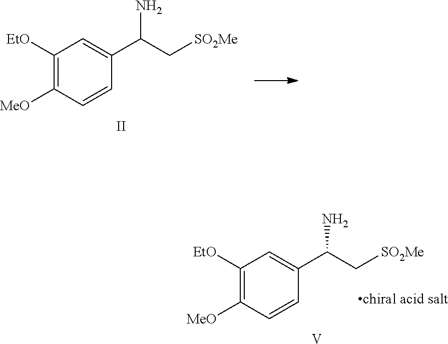

(b) optionally isolating and purifying chiral acid salt of aminosulfone of formula (V);

(c) treating chiral acid salt of aminosulfone of formula (V) with base in suitable solvent to form chiral aminosulfone of formula (Va);

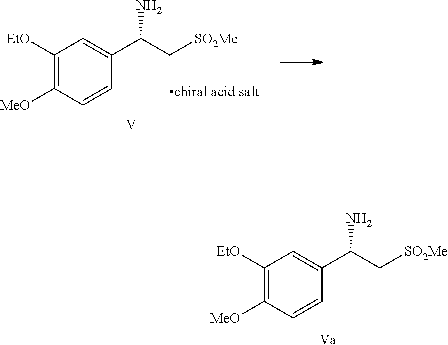

(d) optionally isolating and purifying chiral aminosulfone of formula (Va).

In third embodiment, the present application provides a process for preparation of apremilast of formula (I) or its stereoisomers thereof

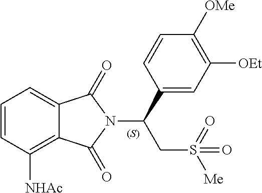

which comprises:

(a) contacting 1-(3-Ethoxy-4-methoxy-phenyl)-2-methanesulfonyl-ethylamine of formula (II) with a chiral acid in presence of a suitable solvent to form a chiral acid salt of 1-(3-Ethoxy-4-methoxy-phenyl)-2-methanesulfonyl-ethylamine of formula (V);

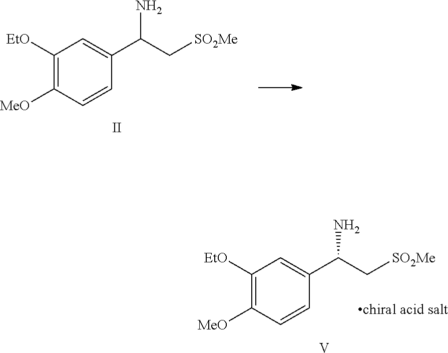

(b) optionally isolating and purifying chiral acid salt of aminosulfone of formula (V);

(c) optionally treating chiral acid salt of aminosulfone of formula (V) with base to form chiral aminosulfone of formula (Va);

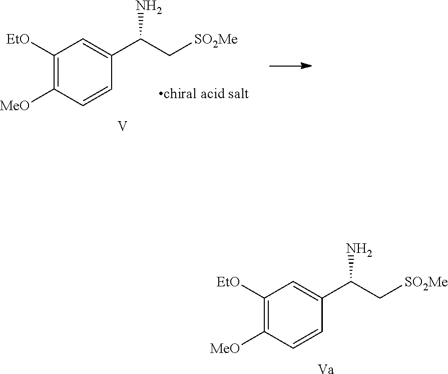

(d) contacting the chiral acid salt of 1-(3-Ethoxy-4-methoxy-phenyl)-2-methanesulfonyl-ethylamine of formula (V) or chiral aminosulfone of formula (Va) with Dioxo-1,3-dihydro-isobenzofuran-4-yl)-acetamide of formula (VI) in presence of a suitable solvent to provide apremilast of formula (I);

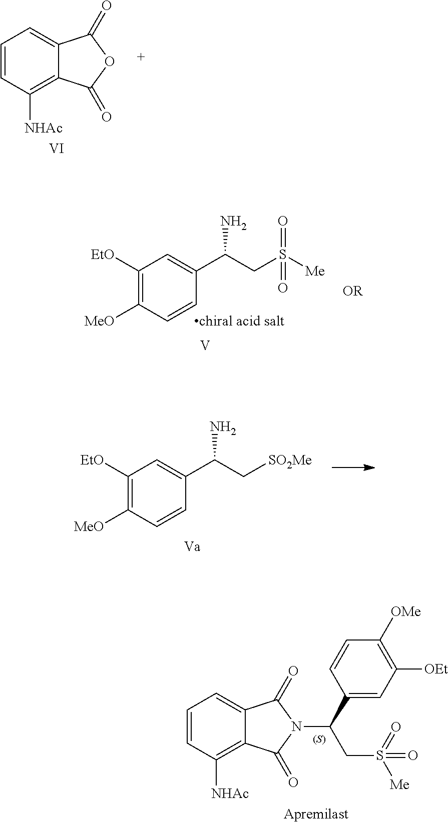

(e) optionally purifying apremilast of formula (I).

In fourth embodiment, the present application provides a process for preparation of apremilast of formula (I) or its stereoisomers thereof:

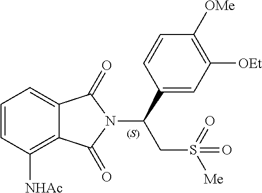

which comprises:

(a) reacting benzonitrile of formula (III) with dimethyl sulfone in the presence of a base in a suitable solvent to provide a compound of formula (IVa), followed by its conversion to provide enamine of formula (IV);

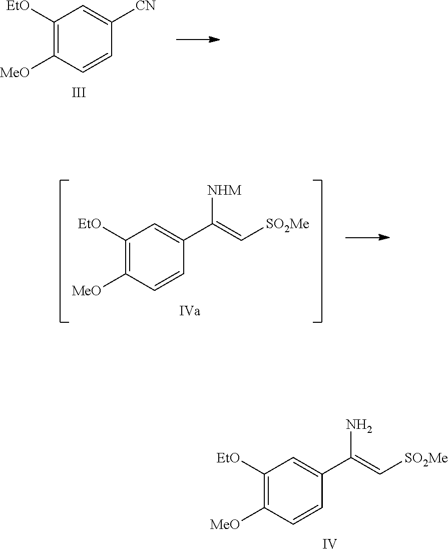

Wherein M=Na, K

(b) reducing enamine of formula (IV) in presence of a suitable solvent to provide aminosulfone of formula (II);

(c) optionally purifying amino sulfone of formula (II);

(d) contacting 1-(3-Ethoxy-4-methoxy-phenyl)-2-methanesulfonyl-ethylamine of formula (II) with a chiral acid in presence of a suitable solvent to form a chiral acid salt of 1-(3-Ethoxy-4-methoxy-phenyl)-2-methanesulfonyl-ethylamine of formula (V);

(e) optionally isolating and purifying chiral acid salt of aminosulfone of formula (V);

(f) optionally treating chiral acid salt of aminosulfone of formula (V) with base to form chiral aminosulfone of formula (Va);

(g) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of a suitable solvent to provide apremilast of formula (I);

(h) optionally purifying apremilast of formula (I).

In fifth embodiment, the present application provides crystalline form of chiral aminosulfone of formula (Va) characterized by its powder X-ray diffraction (PXRD) pattern having peaks at about 5.97, 17.81, 19.85 and 26.07±0.2 degrees 2θ. In embodiments, the present application provides crystalline form of chiral aminosulfone of formula (Va) characterized by its PXRD pattern having additional peaks located at about 11.88, 15.88, 21.96 and 26.72±0.2 degrees 2θ. Still in other embodiment, the present application provides crystalline form of chiral aminosulfone of formula (Va) characterized by its PXRD pattern having additional peaks located at about 12.10, 20.72 and 22.18±0.2 degrees 2θ.

In sixth embodiment, the present application provides crystalline form of chiral aminosulfone of formula (Va) characterized by its powder X-ray diffraction (PXRD) pattern having peaks at about 5.97, 11.88, 12.10, 15.88, 17.81, 19.85, 20.72, 21.96, 22.18, 26.07 and 26.72±0.2 degrees 2θ.

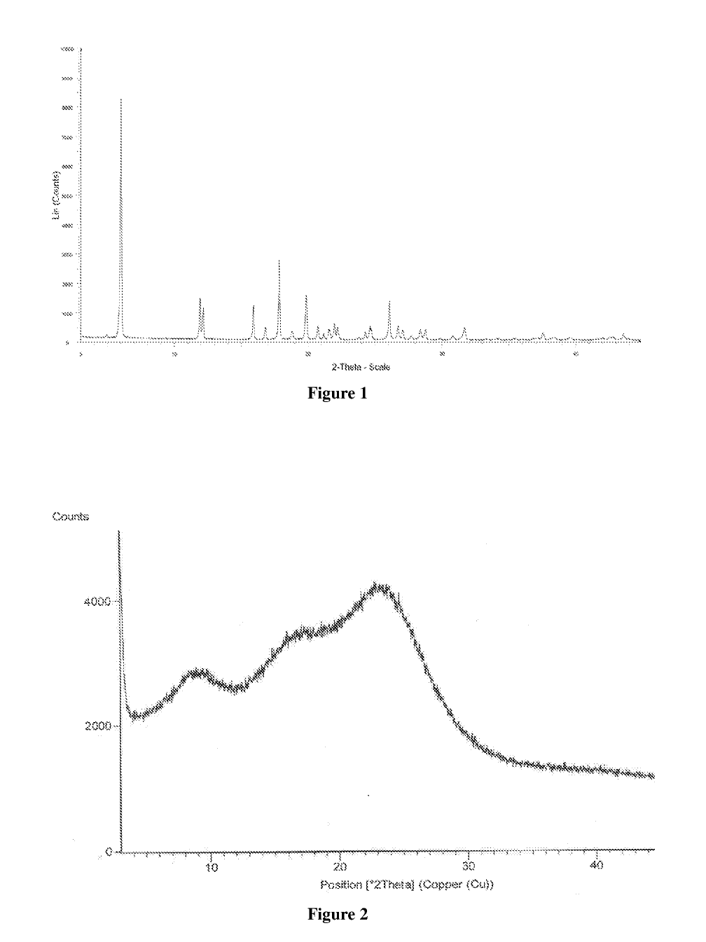

In seventh embodiment, the present application provides crystalline form of chiral aminosulfone of formula (Va) that can be characterized by a PXRD pattern having peaks located substantially as illustrated in the pattern of FIG. 1.

In eighth embodiment, the present application provides a process for the preparation of racemic aminosulfone of formula (II) and its pharmaceutically acceptable salts

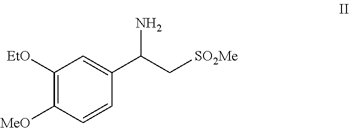 [SMILES: CCOc1cc(C(N)CS(C)(=O)=O)ccc1OC]

which comprises:

(a) reacting of (R)-1-(3-ethoxy-4-methoxy-phenyl)-2-methanesulfonyl-ethylamine of formula (Vb) and its pharmaceutically acceptable salts with a halogenating reagent in the presence of a suitable solvent to provide a halogenated amine of formula (VII);

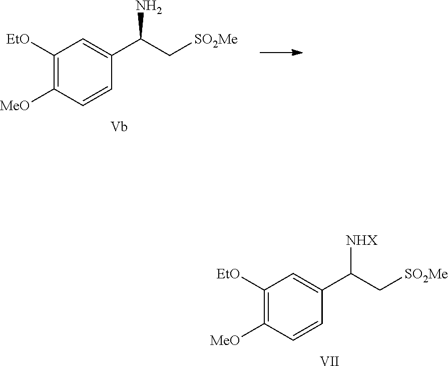

wherein X=Cl, F, Br, I

(b) treating halogenated amine of formula (VII) in presence of a suitable base in a suitable solvent to provide enamine of formula (IV);

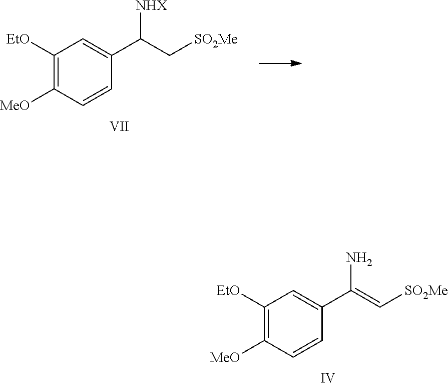

wherein X=Cl, F, Br, I

(c) converting the enamine of formula (IV) to racemic aminosulfone of formula (II) in presence of reducing agent and a suitable solvent;

(d) optionally purifying racemic amino sulfone of formula (II).

In ninth embodiment, the present application provides a process for preparation of apremilast of formula (I) or its stereoisomers thereof:

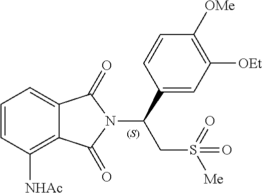

which comprises:

(a) reacting of (R)-1-(3-ethoxy-4-methoxy-phenyl)-2-methanesulfonyl-ethylamine of formula (Vb) and its pharmaceutically acceptable salts with a halogenating reagent in the presence of a suitable solvent to provide a halogenated amine of formula (VII);

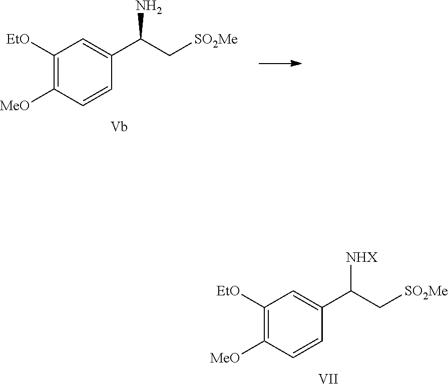

wherein X=Cl, F, Br, I

(b) treating halogenated amine of formula (VII) in presence of a suitable base in a suitable solvent to provide enamine of formula (IV);

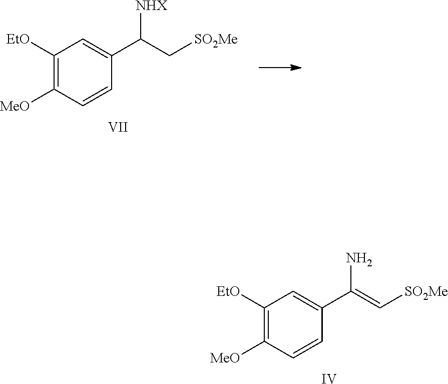

wherein X=Cl, F, Br, I

(c) converting the enamine of formula (IV) to racemic aminosulfone of formula (II) in presence of reducing agent and a suitable solvent;

(d) optionally purifying racemic amino sulfone of formula (II).

(e) converting the racemic amino sulfone of formula (II) to apremilast of formula (I);

(f) optionally purifying apremilast of formula (I).

In tenth embodiment, the present application provides novel compound of formula (VII)

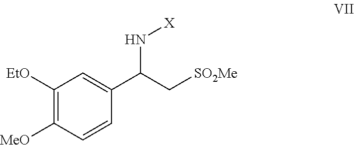

wherein X=Cl, F, Br, I

In eleventh embodiment, the present application provides a process for preparation of apremilast of formula (I) or its stereoisomers thereof which comprises:

(a) contacting the chiral acid salt of 1-(3-Ethoxy-4-methoxy-phenyl)-2-methanesulfonyl-ethylamine of formula (V) or chiral amino sulfone of formula (Va) with N-(1,3-Dioxo-1,3-dihydro-isobenzofuran-4-yl)-acetamide of formula (VI) in presence of mixture of ketonic solvent and polar solvent to provide apremilast of formula (I);

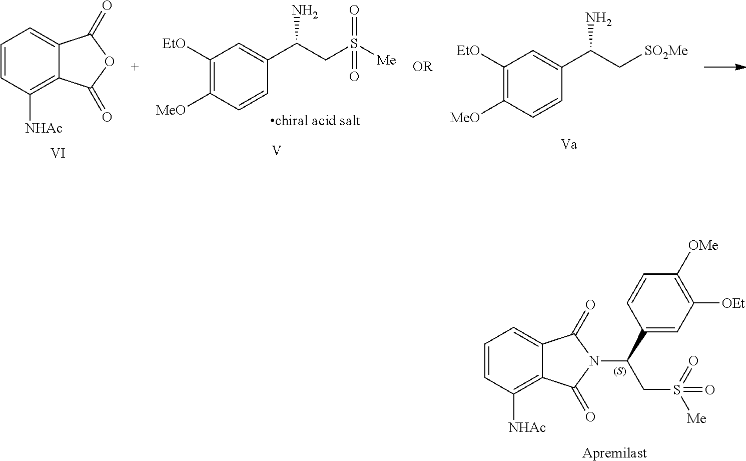

(b) optionally purifying apremilast of formula (I).

In twelveth embodiment, the present application provides a process for preparation of crystalline form B of apremilast of formula (I) or its stereoisomers thereof:

which comprises:

(a) contacting the chiral acid salt of 1-(3-Ethoxy-4-methoxy-phenyl)-2-methanesulfonyl-ethylamine of formula (V) or chiral amino sulfone of formula (Va) with N-(1,3-Dioxo-1,3-dihydro-isobenzofuran-4-yl)-acetamide of formula (VI) in presence of mixture of ketonic solvent and polar solvent to provide apremilast of formula (I);

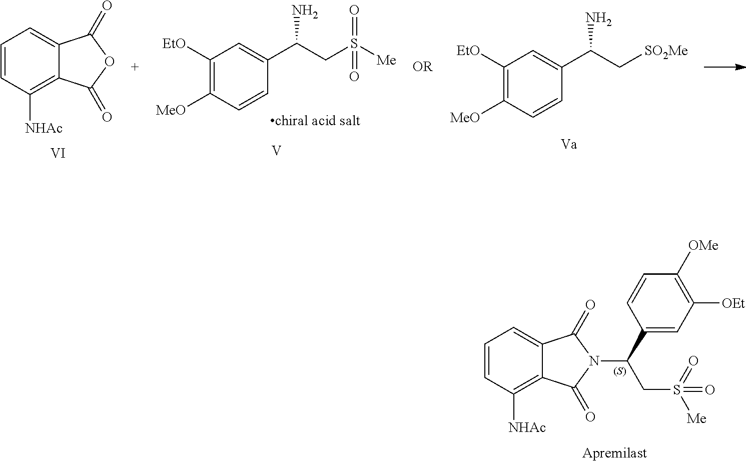

(b) optionally isolating and purifying apremilast;

(c) converting the apremilast obtained in step (b) to crystalline form B of apremilast;

(d) optionally isolating and purifying crystalline form B of apremilast.

In thirteenth embodiment, the present application provides process for preparing amorphous form of apremilast comprising:

a) dissolving apremilast in a suitable solvent or mixture thereof;

b) optionally, heating the solution of step (a);

c) adding water as an anti-solvent to the solution of apremilast; or adding solution of apremilast to water;

d) isolating the solid;

e) optionally, drying the product at suitable temperature.

In fourteenth embodiment, the present invention provides desoxo impurity of Apremilast, which is designated as Impurity M and having the following structure:

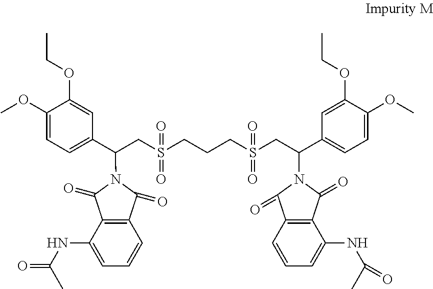

In fifteenth embodiment, the present application provides pharmaceutical compositions comprising apremilast of formula (I) prepared according to processes of the present application together with one or more pharmaceutically acceptable excipient, carrier and diluents.

### DETAILED DESCRIPTION

The present application provides novel synthetic processes for obtaining Apremilast of formula (I) and its related intermediates.

In first embodiment, the present application provides a process for preparation of aminosulfone of formula (II) or its stereo isomers and their pharmaceutically acceptable salts

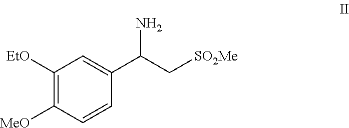 [SMILES: CCOc1cc(C(N)CS(C)(=O)=O)ccc1OC]

which comprises:

(a) reacting benzonitrile of formula (III) with dimethyl sulfone in the presence of a base in a suitable solvent to provide a compound of formula (IVa), followed by its conversion to provide enamine of formula (IV);

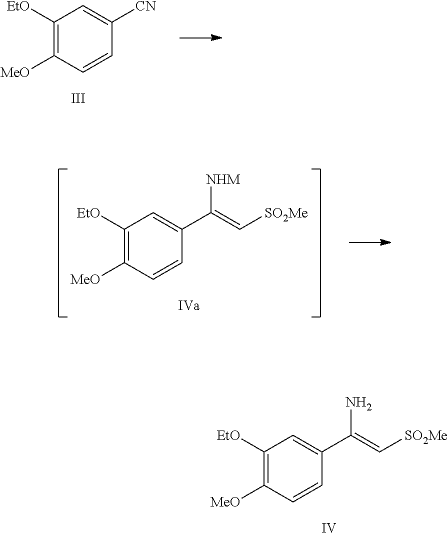

Wherein M=Na, K

(b) reducing enamine of formula (IV) in presence of a suitable solvent to provide aminosulfone of formula (II);

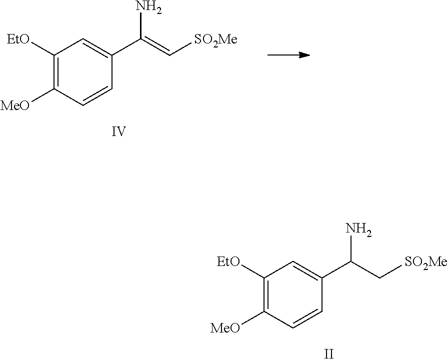

(c) optionally purifying amino sulfone of formula (II).

Step (a) may be carried out in the presence of one or more suitable bases. Suitable base that may be used in step (a) include, but are not limited sodium amide, potassium amide, C₁-C₂₀ alkoxide of sodium, C₁-C₂₀ alkoxide of potassium, C₁-C₂₀ alkoxide of magnesium, sodium hydride, potassium hydride and the like.

Step (a) may be carried out in one or more suitable solvents. Suitable solvent that may be used in step (a) include, but are not limited to ether solvents, such as, for example, diethyl ether, diisopropyl ether, tert-butyl methyl ether, dibutyl ether, tetrahydrofuran, 2-methyl tetrahydrofuran,1,2-dimethoxyethane, 2-methoxyethanol, 2-ethoxyethanol, anisole, 1, 4-dioxane, or the like; ketone solvents, such as, for example, acetone, dialkyl ketone or the like; aromatic hydrocarbon solvents, such as, for example, toluene, xylene, chlorobenzene, tetralin, or the like; nitrile solvents, such as, for example, acetonitrile, propionitrile, or the like; alcohol solvents, such as, for example, methanol, ethanol, isopropanol or the like; ester solvents, such as, for example, ethyl formate, methyl acetate, ethyl acetate, propyl acetate, butyl acetate, methyl propanoate, ethyl propanoate, methyl butanoate, ethyl butanoate, or the like or amide solvents, such as, for example, dimethylacetamide, dimethylformamide or the like, dimethylsulfoxide or mixtures thereof.

In an embodiment, the salt of enamine of compound of formula (IVa) may be directly subjected to reduction without converting its corresponding enamine of formula (IV) to afford amino sulfone of formula (II). The substantially similar reaction conditions may be adopted to that of free base of enamine compound (IV).

Any reducing agent known in the art for reducing an enamine to an amine can be used for the reduction in step (b). The reducing agent that may be used in step (b) include, but are not limited to sodium triacetoxyborohydride, sodium borohydride, sodium cyano borohydride, Palladium, Raney-nickel and the like.

The reduction in step (b) can occur in the presence of an acid such as, but not limited to, acetic acid, methanesulfonic acid, trifluoroacetic acid, 4-(trifluoromethyl)benzoic acid, p-toluenesulfonic acid, hydrochloric acid, nitric acid, sulfuric acid, phosphoric acid, citric acid, tartaric acid, benzene sulfonic acid and the like.

Step (c) which involves the isolation and purification of compound of formula (II) or its pharmaceutically acceptable salt can be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

The temperature at which the above steps may be carried out in between about 0° C. and about 100° C., preferably at about 5° C. and about 60° C., based on the solvent or mixture of solvent used in particular step.

In second embodiment, the present application provides a process for the preparation of formula (Va) or its stereoisomers thereof

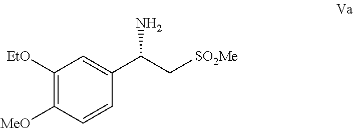 [SMILES: CCOc1cc([C@H](N)CS(C)(=O)=O)ccc1OC]

which comprises:

(a) contacting racemic 1-(3-ethoxy-4-methoxy-phenyl)-2-methanesulfonyl-ethylamine of formula (II) with a chiral acid in presence of a suitable solvent to form a chiral acid salt of 1-(3-ethoxy-4-methoxy-phenyl)-2-methanesulfonyl-ethylamine of formula (V);

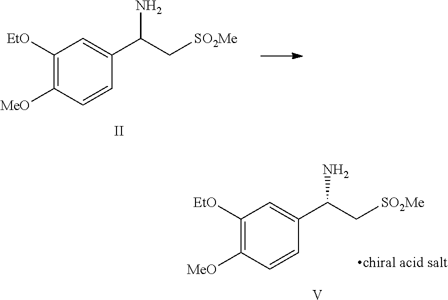

(b) optionally isolating and purifying chiral acid salt of aminosulfone of formula (V);

(c) treating chiral acid salt of aminosulfone of formula (V) with base in suitable solvent to form chiral aminosulfone of formula (Va);

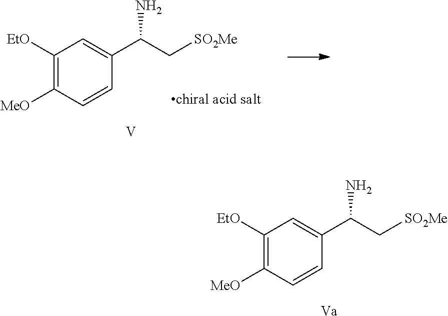

(d) optionally isolating and purifying chiral aminosulfone of formula (Va).

Step (a) may be carried out in one or more suitable solvents. Suitable solvent that may be used in step (a) include, but are not limited to ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1, 4-dioxane, and the like; ketone solvents, such as, for example, acetone, methyl ethyl ketone and the like; aromatic hydrocarbon solvents, such as, for example, toluene, xylene, chlorobenzene, tetralin, and the like; nitrile solvents, such as, for example, acetonitrile, propionitrile, and the like; alcohol solvents, such as, for example, methanol, ethanol, isopropanol and the like; ester solvents, such as, for example, ethyl formate, methyl acetate, ethyl acetate, propyl acetate, butyl acetate, methyl propanoate, ethyl propanoate, methyl butanoate, ethyl butanoate, and the like or amide solvents, such as, for example, dimethylacetamide, dimethylformamide, N-methylpyrrolidone and the like or acids such formic acid, acetic acid, propionic acid; water or mixtures thereof.

Suitable chiral acids that may be used in step (a) include, but are not limited to individual enantiomers of 10-camphorsulfonic acid, camphoric acid, methoxyacetic acid, tartaric acid, diacetyltartaric acid, di-toluoyl tartaric acid, dibenzoyl tartaric acid, mandelic acid, derivatives of mandelic acid such as acetyl mandelic acid, propyl mandelic acid, lactic acid, ibuprofen, malic acid, pyrrolidone-5-carboxylic acid, naproxen, and the like. Specifically suitable chiral acids that may be used in step (a) include, but are not limited to tartaric acid, dibenzoyl tartaric acid and di toluoyl tartaric acid. More specifically the suitable chiral acid may be dibenzoyl tartaric acid. The resolution may also be carried out under any of the Pope-Peachey resolution conditions or any conventional method of resolution. For Pope Peachey resolution, an organic or inorganic acid in water along with chiral resoluting agent can be used for the chiral resolution process at any of the mole ratios between chiral acid, organic/inorganic acid and water.

Step (b) which involves the isolation and purification of compound of formula (V) or its pharmaceutically acceptable salt can be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures. Any suitable solvent that is capable to dissolve the salt may be used for the purification to increase the chiral purity to a desired level.

Step (c) may be carried out in the presence of one or more suitable bases and one or more suitable solvents. Suitable base that may be used in step (c) include, but are not limited to organic bases like pyridine, piperidine, pyrimidine, triethylamine, diethylamine, diisopropyl ethylamine, 1,1,3,3-tetramethylguanidine, DBU, DABCO etc, inorganic bases like metal carbonates such as sodium carbonate, potassium carbonate; metal bicarbonates such as sodium bicarbonate, potassium bicarbonate; metal hydroxide like sodium hydroxide, potassium hydroxide, lithium hydroxide, calcium hydroxide and the like.

Step (c) may be carried out in one or more suitable solvents. Suitable solvent that may be used in step (c) include, but are not limited to but are not limited to aromatic hydrocarbon solvents, such as, for example, toluene, xylene, chlorobenzene, tetralin, and the like; halogenated hydrocarbons such as dichloromethane, chloroform and the like; alcoholic solvents like methanol, ethanol, isopropyl alcohol and the like; water, and mixtures thereof.

Step (d) which involves the isolation and purification of compound of formula (Va) or its pharmaceutically acceptable salt can be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

Optionally, the resulted chiral amino sulfone of formula (Va) may be subjected to purification in one or more suitable solvents.

The temperature at which the above steps may be carried out in between about 0° C. and about 100° C., preferably at about 25° C. and about 70° C., based on the solvent or mixture of solvent used in particular step.

In third embodiment, the present application provides a process for preparation of apremilast of formula (I) or its stereoisomers thereof

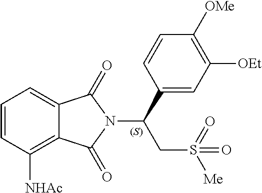

which comprises:

(a) contacting 1-(3-ethoxy-4-methoxy-phenyl)-2-methanesulfonyl-ethylamine of formula (II) with a chiral acid in presence of a suitable solvent to form a chiral acid salt of 1-(3-ethoxy-4-methoxyphenyl)-2-methanesulfonyl-ethylamine of formula (V);

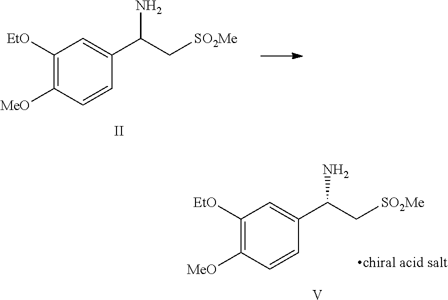

(b) optionally isolating and purifying chiral acid salt of aminosulfone of formula (V);

(c) optionally treating chiral acid salt of aminosulfone of formula (V) with base to form chiral aminosulfone of formula (Va);

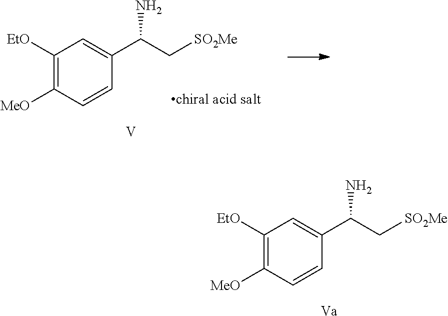

(d) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of a suitable solvent to provide apremilast of formula (I);

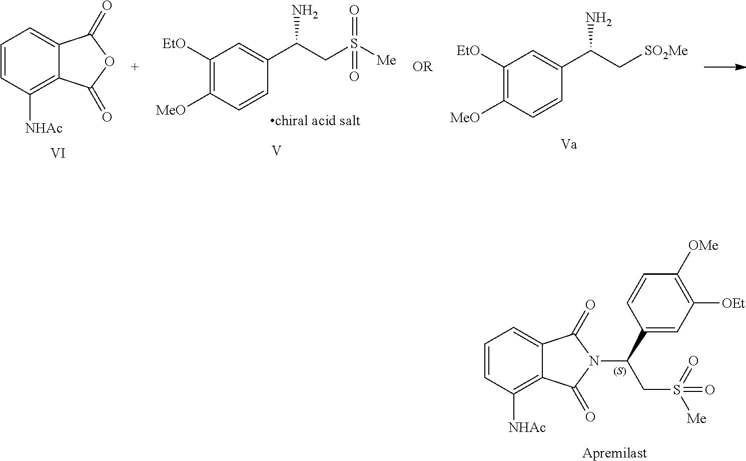

(e) optionally purifying apremilast of formula (I).

The reagents, solvents and reaction conditions for steps (a), (b) and (c) may be selected from one or more suitable reagents, solvents and process conditions as described in the steps of first and second embodiments of the present invention.

Step (d) may be carried out in one or more suitable solvents. Suitable solvent that may be used in step (c) include, but are not limited to ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1, 4-dioxane, or the like; ketone solvents, such as, for example, acetone, dialkyl ketone or the like; aromatic hydrocarbon solvents, such as, for example, toluene, xylene, chlorobenzene, tetralin, or the like; nitrile solvents, such as, for example, acetonitrile, propionitrile, or the like; alcohol solvents, such as, for example, methanol, ethanol, isopropanol or the like; ester solvents, such as, for example, ethyl formate, methyl acetate, ethyl acetate, propyl acetate, butyl acetate, methyl propanoate, ethyl propanoate, methyl butanoate, ethyl butanoate, or the like or amide solvents, such as, for example, dimethylacetamide, dimethylformamide or acid like formic acid, acetic acid, propionic acid, butanoic acid, methane sulphonic acid, benzene sulphonic acid, or anhydride like acetic anhydride, propionic anhydride in combination with water or mixtures thereof.

Step (e) which involves the isolation and purification of compound of formula (I) can be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

The temperature at which the above steps may be carried out in between about 0° C. and about 100° C., preferably at about 25° C. and about 100° C., based on the solvent or mixture of solvent used in particular step.

In fourth embodiment, the present application provides a process for preparation of apremilast of formula (I) or its stereoisomers thereof:

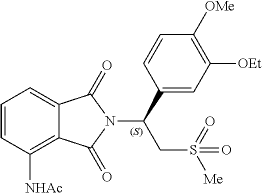

which comprises:

(a) reacting benzonitrile of formula (III) with dimethyl sulfone in the presence of a base in a suitable solvent to provide a compound of formula (IVa), followed by its conversion to provide enamine of formula (IV);

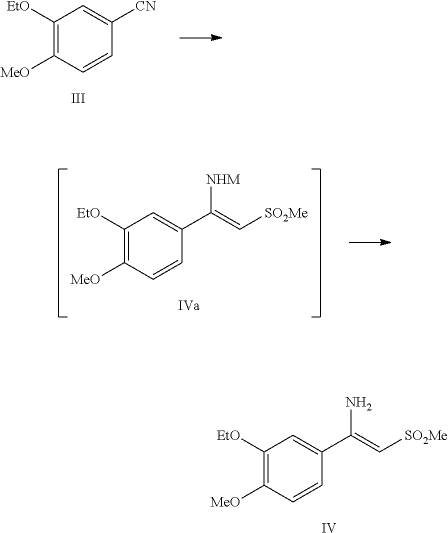

Wherein M=Na, K

(c) reducing enamine of formula (IV) in presence of a suitable solvent to provide aminosulfone of formula (II);

(c) optionally purifying amino sulfone of formula (II);

(d) contacting 1-(3-ethoxy-4-methoxy-phenyl)-2-methanesulfonyl-ethylamine of formula (II) with a chiral acid in presence of a suitable solvent to form a chiral acid salt of 1-(3-Ethoxy-4-methoxy-phenyl)-2-methanesulfonyl-ethylamine of formula (V);

(e) optionally isolating and purifying chiral acid salt of aminosulfone of formula (V);

(f) optionally treating chiral acid salt of aminosulfone of formula (V) with base to form chiral aminosulfone of formula (Va);

(g) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of a suitable solvent to provide apremilast of formula (I);

(h) optionally purifying apremilast of formula (I).

The reagents, solvents and reaction conditions for steps (a) to (h) may be selected from one or more suitable reagents, solvents and process conditions as described in the steps of the first, second and third embodiments of the present invention.

In fifth embodiment, the present application provides crystalline form of chiral aminosulfone of formula (Va) characterized by its powder X-ray diffraction (PXRD) pattern having peaks at about 5.97, 17.81, 19.85 and 26.07±0.2 degrees 2θ. In embodiments, the present application provides crystalline form of chiral aminosulfone of formula (Va) characterized by its PXRD pattern having additional peaks located at about 11.88, 15.88, 21.96 and 26.72±0.2 degrees 2θ. Still in other embodiment, the present application provides crystalline form of chiral aminosulfone of formula (Va) characterized by its PXRD pattern having additional peaks located at about 12.10, 20.72 and 22.18±0.2 degrees 2θ.

In sixth embodiment, the present application provides crystalline form of chiral aminosulfone of formula (Va) characterized by its powder X-ray diffraction (PXRD) pattern having peaks at about 5.97, 11.88, 12.10, 15.88, 17.81, 19.85, 20.72, 21.96, 22.18, 26.07 and 26.72±0.2 degrees 2θ.

In seventh embodiment, the present application provides crystalline form of chiral aminosulfone of formula (Va) that can be characterized by a PXRD pattern having peaks located substantially as illustrated in the pattern of FIG. 1.

The PXRD data reported herein is obtained by using a PANalytical X-ray Diffractometer, with copper Kα radiation. Chiral amino sulfone of formula (Va) and/or apremilast of formula (I) obtained in the present invention is having more than 95% chemical and chiral purity.

In eighth embodiment, the present application provides a process for the preparation of racemic aminosulfone of formula (II) and its pharmaceutically acceptable salts

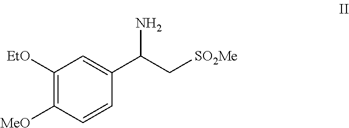 [SMILES: CCOc1cc(C(N)CS(C)(=O)=O)ccc1OC]

which comprises:

(a) reacting of (R)-1-(3-ethoxy-4-methoxy-phenyl)-2-methanesulfonyl-ethylamine of formula (Vb) and its pharmaceutically acceptable salts with a halogenating reagent in the presence of a suitable solvent to provide a halogenated amine of formula (VII);

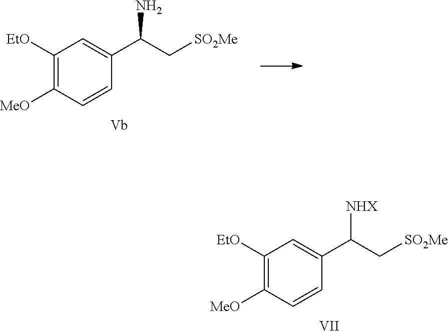

wherein X=Cl, F, Br, I

(b) treating halogenated amine of formula (VII) in presence of a suitable base in a suitable solvent to provide enamine of formula (IV);

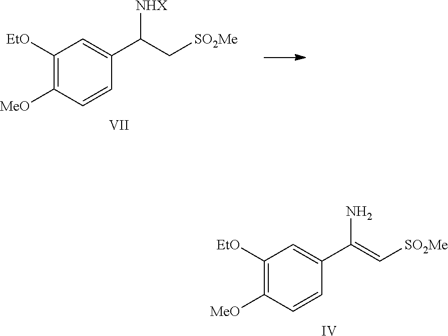

wherein X=Cl, F, Br, I

(c) converting the enamine of formula (IV) to racemic aminosulfone of formula (II) in presence of reducing agent and a suitable solvent;

(d) optionally purifying racemic amino sulfone of formula (II).

Suitable halogenating reagents that may be used in step (a) include, but are not limited to Sodium dichloroisocyanurate (NaDCC), trichloroisocyanuric acid, N,N′-dichlorobis(2,4,6-trichlorophenyl)urea, N-chlorosuccinimide, N-bromosuccinimide, sodium hypochlorite, sodium hypobromite and the like.

Step (a) may be carried out in one or more suitable solvents. Suitable solvent that may be used in step (a) include, but are not limited to ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1, 4-dioxane, or the like; aromatic hydrocarbon solvents, such as, for example, toluene, xylene, chlorobenzene, tetralin, or the like; chlorinated hydrocarbon solvents, such as chloroform, dichloromethane or the like; alcohol solvents, such as, for example, methanol, ethanol, isopropanol or the like; water and mixtures thereof.

Suitable bases that may be used in step (b) include, but are not limited to organic bases such as, 2,4,6-collidine, 2,6-di-tert-butyl-4-methylpyridine, 1-diethylamino-2-propanol, N-ethylamino-2-propanol, N-ethyldiisopropylamine, 4-ethylmorpholine, 1-ethylpiperidine, 2,6-lutidine, N-methylmorpholine, 1-methylpiperidine, tribenzylamine, triethylamine, DBU, pyridine, LDA, NaHMDS, KHMDS, sodium hydride, potassium hydride and the like. Suitable inorganic bases include, but are not limited to alkali hydrides, such as, for example, sodium hydride, potassium hydride or the like; alkali metal hydroxides, such as, for example, lithium hydroxide, sodium hydroxide, potassium hydroxide, and cesium hydroxide or the like; alkaline earth metal hydroxides, such as, for example, barium hydroxide, strontium hydroxide, magnesium hydroxide, calcium hydroxide, or the like; alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, sodium bicarbonate, potassium bicarbonate, or the like.

Step (b) may be carried out in one or more suitable solvents. Suitable solvent that may be used in step (b) include, but are not limited to ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1, 4-dioxane, or the like; aromatic hydrocarbon solvents, such as, for example, toluene, xylene, chlorobenzene, tetralin, or the like; chlorinated hydrocarbon solvents, such as chloroform, dichloromethane or the like; alcohol solvents, such as, for example, methanol, ethanol, isopropanol or the like and mixtures thereof.

Step (c) may be carried out in one or more suitable reducing agents. Suitable reducing agents that may be used in step (c) include, but are not limited to sodium borohydride, lithium borohydride, sodium cyanoborohydride, di-isobutyl aluminum hydride and the like.

Step (c) may be carried out in one or more suitable solvents. Suitable solvent that may be used in step (a) include, but are not limited to ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1, 4-dioxane, or the like; aromatic hydrocarbon solvents, such as, for example, toluene, xylene, chlorobenzene, tetralin, or the like; chlorinated hydrocarbon solvents such as chloroform, dichloromethane or the like; alcohol solvents, such as, for example, methanol, ethanol, isopropanol or the like; water and mixtures thereof.

Step (d) which involves the isolation and purification of compound of formula (II) or its pharmaceutically acceptable salt can be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

The temperature at which the above steps may be carried out in between about −20° C. and about 100° C., preferably at about 0° C. and about 25° C., based on the solvent or mixture of solvent used in the particular step.

In ninth embodiment, the present application provides a process for preparation of apremilast of formula (I) or its stereoisomers thereof:

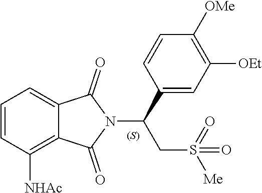

which comprises:

(a) reacting of (R)-1-(3-ethoxy-4-methoxy-phenyl)-2-methanesulfonyl-ethylamine of formula (Vb) and its pharmaceutically acceptable salts with a halogenating reagent in the presence of a suitable solvent to provide a halogenated amine of formula (VII);

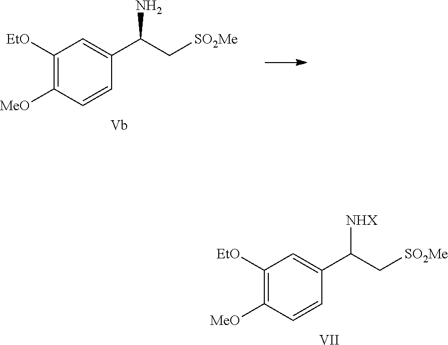

wherein X=Cl, F, Br, I

(b) treating halogenated amine of formula (VII) in presence of a suitable base in a suitable solvent to provide enamine of formula (IV);

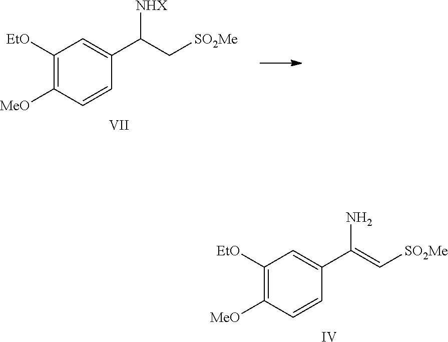

wherein X=Cl, F, Br, I

(c) converting the enamine of formula (IV) to racemic aminosulfone of formula (II) in presence of reducing agent and a suitable solvent;

(d) optionally purifying racemic amino sulfone of formula (II).

(e) converting the racemic amino sulfone of formula (II) to apremilast of formula (I);

(f) optionally purifying apremilast of formula (I).

The reagents, solvents and reaction conditions for steps (a) to (d) may be selected from one or more suitable reagents, solvents and process conditions as described in the steps (a) to (d) of the eighth embodiment of the present invention.

Racemic aminosulfone of formula (II) obtained in the present invention may be converted to apremilast of formula (I) by methods known in the art. The intermediates obtained in the present invention may be isolated or used directly in the next step.

In tenth embodiment, the present application provides novel compound of formula (VII)

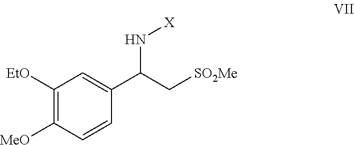

wherein X=Cl, F, Br, I

The present invention further includes the use of novel compound of formula (VII) for the preparation of apremilast or its intermediates.

In eleventh embodiment, the present application provides a process for preparation of apremilast of formula (I) or its stereoisomers thereof which comprises:

(a) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of mixture of ketonic solvent and polar solvent to provide apremilast of formula (I);

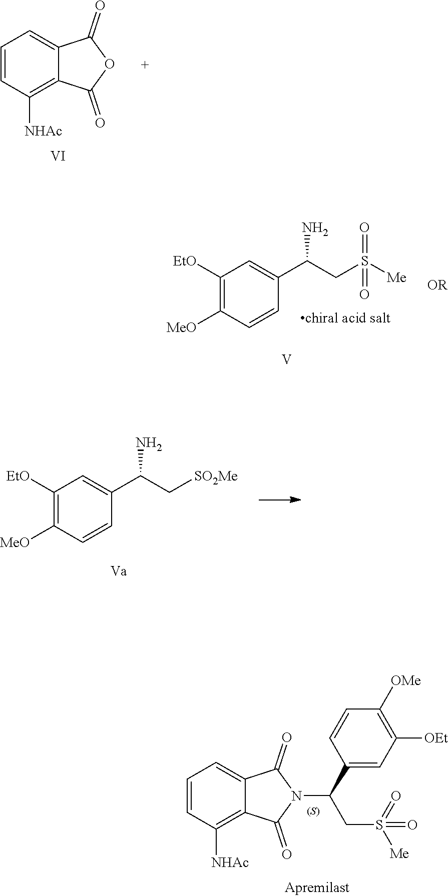

(b) optionally purifying apremilast of formula (I).

Suitable ketone solvent that may be used in step (a) include, but are not limited to acetone, dialkyl ketone such as ethyl methyl ketone, methyl isobutyl ketone or the like; or mixtures thereof.

Suitable polar solvent that may be used in step (a) include, but are not limited to acid such as acetic acid, formic acid and the like; ethers such as tetrahydrofuran, diethyl ether and the like; nitriles such as acetonitrile, propionitrile and the like; esters such as ethyl acetate, isopropyl acetate and the like; alcohols such as methanol, ethanol and the like; amides such as dimethylformamide, dimethylacetamide and the like; dimethylsulfoxide, water or mixtures thereof.

In an embodiment, the ratio of ketone to polar solvent in above step lies in the ratio of 25:1 v/v, preferably at about 20:1 v/v.

Suitable chiral acids that may be used in step (a) include, but are not limited to individual enantiomers of 10-camphorsulfonic acid, camphoric acid, alpha-bromocamphoric acid, methoxyacetic acid, tartaric acid, diacetyltartaric acid, di toluoyl tartaric acid, dibenzoyl tartaric acid, mandelic acid, lactic acid, ibuprofen, malic acid, pyrrolidone-5-carboxylic acid, naproxen, 3-(2-amino-2-oxoethyl)-5-methylhexanoic acid, and the like.

Step (b) which involves the isolation and purification of compound of formula (I) can be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

The temperature at which the above steps may be carried out in between about 0° C. and about 100° C., preferably at about 25° C. and about 100° C., based on the solvent or mixture of solvent used in particular step.

In a particular embodiment, the free base of formula (Va) is an amino sulfone intermediate, which is prepared by following the processes known in the literature or by neutralizing the chiral acid salt of 1-(3-Ethoxy-4-methoxy-phenyl)-2-methanesulfonyl-ethylamine of formula (Vb) with a base to afford racemic amino sulfone.

In twelveth embodiment, the present application provides a process for preparation of crystalline form B of apremilast of formula (I) or its stereoisomers thereof:

which comprises:

(a) contacting the chiral acid salt of 1-(3-Ethoxy-4-methoxy-phenyl)-2-methanesulfonyl-ethylamine of formula (V) or chiral amino sulfone of formula (Va) with N-(1,3-Dioxo-1,3-dihydro-isobenzofuran-4-yl)-acetamide of formula (VI) in presence of mixture of ketonic solvent and polar solvent to provide apremilast of formula (I);

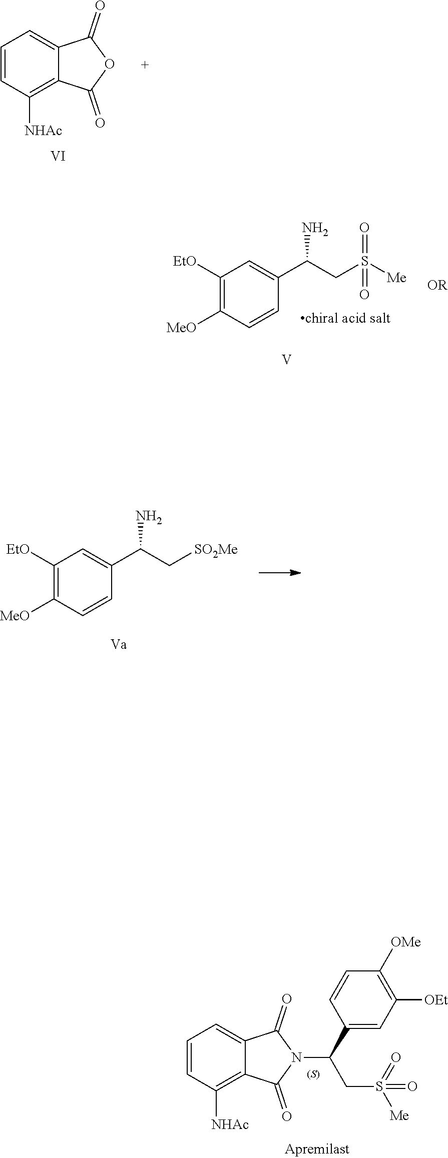

(b) optionally isolating and purifying apremilast;

(c) converting the apremilast obtained in step (b) to crystalline form B of apremilast;

(d) optionally isolating and purifying crystalline form B of apremilast.

The reagents, solvents and reaction conditions for step (a) may be selected from one or more suitable reagents, solvents and process conditions as described in the steps of the eleventh embodiment of the present invention.

The crystalline form obtained in step b) of the instant invention can be any single crystalline form or mixture containing one or more crystalline forms of apremilast known in the art.

The isolation of steps b) and d) can be effected, if desired, by any suitable separation method such as precipitation, filtration, centrifugation, extraction, acid-base treatment, conventional isolation and refining means such as concentration, concentration under reduced pressure or by a combination of these procedures.

The temperature at which the above steps may be carried out in between about 0° C. and about 100° C., preferably at about 25° C. and about 100° C., based on the solvent or mixture of solvent used in particular step.

The apremilast obtained in step (b) of the present invention may be dried by any of the known drying methods and/or may be further purified by the known purification techniques.

In an embodiment, the crystalline form B of apremilast may be added as a seed crystal in step (c) of the present invention. The quantity of seed crystal may be used from about 0.5 wt % to about 50 wt %, preferably the quantity of seed crystal may be about 0.5 wt % over input material.

In one of the embodiment, the reaction mass obtained in step (c) of the present invention may be stirred at different temperature ranges for suitable time period. Particularly, the reaction mass obtained in step (c) of the present invention may be stirred at a temperature of about 70-95° C. for about 2-4 hours, followed by at about 40-65° C. for about 10-20 hours and further at about 25-35° C. for about 10-30 hours. The stirring temperature and time period may be varied based on the conversion of apremilast obtained in step (b) to crystalline form B of apremilast.

In one of the aspect of the present invention, the apremilast obtained in step (b) may be crystalline form A or a mixture of crystalline form A with other known polymorphic forms of apremilast and preferably, the polymorphic conversion may be monitored by X-ray diffraction analysis.

In thirteenth embodiment, the present application provides process for preparing amorphous form of apremilast comprising:

a) dissolving apremilast in a suitable solvent or mixture thereof;

b) optionally, heating the solution of step (a);

c) adding water as an anti-solvent to the solution of apremilast; or adding solution of apremilast to water;

d) isolating the solid;

e) optionally, drying the product at suitable temperature.

Suitable solvents of step a) for dissolving apremilast include, but are not limited to dimethylformamide; dimethylacetamide; dimethyl sulphoxide; nitriles such as acetonitrile, propionitrile and the like; ketones such as acetone, ethyl methyl ketone, methyl isobutyl ketone; ethers such as tetrahydrofuran, dioxane; esters such as ethyl acetate, isopropyl acetate; halogenated hydrocarbons such as chloroform, dichloromethane; alcohols such as methanol, ethanol, propanol, isopropanol; and mixtures thereof.

In an embodiment, water as an anti-solvent may be added to the reaction solution obtained in either step (a) or step (b) of the present invention by drop wise or in a single lot based on the solvent used in step (a) of the present invention. Optionally, the solution obtained in step (a) or step (b) may be added to water.

The temperature at which the above steps may be carried out in between about 20° C. and about 100° C., based on the solvent or mixture of solvent used in particular step.

The isolation of step d) can be effected, if desired, by any suitable separation methods such as precipitation, filtration, centrifugation, extraction, acid-base treatment, conventional isolation and refining means such as concentration, concentration under reduced pressure or by a combination of these procedures.

Drying in the embodiments of the present invention may be suitably carried out by using any of an air tray dryer, vacuum tray dryer, fluidized bed dryer, spin flash dryer, flash dryer, and the like. The drying may be carried out at atmospheric pressure or above, or under reduced pressures, specifically at temperatures less than about 80° C. and more specifically less than about 60° C. The drying may be carried out for any time period required for obtaining a desired product quality, such as from about 30 minutes to about 24 hours, or longer.

The amorphous apremilast obtained in the thirteenth embodiment of the present invention is substantially free of crystalline peaks. In an embodiment, the amorphous form of apremilast obtained in the present invention may have less than about 5 wt % of crystalline apremilast, particularly the amorphous form of apremilast may contain less than about 1 wt % of crystalline apremilast.

The glass-liquid transition or glass transition temperature is the reversible transition in amorphous materials from a hard and relatively brittle state into a molten or rubber-like state. The glass-transition temperature (Tg) is always lower than the melting temperature (Tm) of the crystalline state of the material, if one exists. The glass transition temperature of amorphous apremilast of present invention lies in the range of 76.03° C.-78.83° C.

The amorphous material obtained in the present invention is subjected to humidification studies at different temperature conditions with different relative humidity percentages. In a particular example, the humidification studies were carried out on amorphous apremilast of present invention at about 30° C. and relative humidity of 60% and 90% individually. The amorphous apremilast obtained in the present invention is pure, non-hygroscopic and is stable for about 24 hours at these humidity conditions.

Apremilast of formula (I) obtained in any of the above inventions may optionally be subjected to milling, grinding or sieving techniques to get the required particle size by adopting the procedures known in the art.

In fourteenth embodiment, the present invention provides desoxo impurity of Apremilast, which is designated as Impurity M and having the following structure:

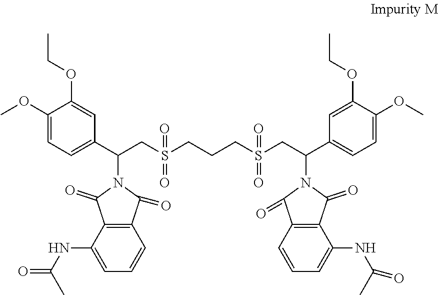

The desoxo impurity of Apremilast (Impurity M) indicates that two apremilast units are linked together by a methylene bridge on the methyl carbon of the chiral amino sulphoxide chain. This impurity formation may occur via the cyclocondensation of 2,2′-(propane-1,3-diyldisulfonyl)bis(1-(3-ethoxy-4-methoxyphenyl)ethan-1-amine) of formula (VIII) with N-(1,3-dioxo-1,3-dihydroisobenzofuran-4-yl)acetamide of formula (VI) in presence of acetic acid in MIBK solvent. The diamine which leads to the formation of Desoxo impurity of Apremilast (Impurity M). The impurity M of Apremilast is characterized by ¹H-NMR, ¹³C-NMR, IR and Mass spectra and other relevant 2D NMR studies.

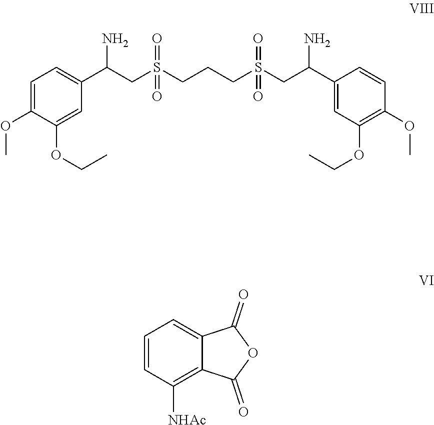 [SMILES: CCOc1cc(C(N)CS(=O)(=O)CCCS(=O)(=O)CC(N)c2ccc(OC)c(OCC)c2)ccc1OC ; CC(=O)Nc1cccc2c1C(=O)OC2=O]

The present invention provides Apremilast (I) substantially free of Impurity M. The present invention further provides Apremilast (I) having 0.01 to about 0.15% w/w of Impurity M by area percentage in HPLC. In another embodiment, the invention provides a pharmaceutical composition comprising Apremilast (I) having 0.01 to about 0.15% w/w of Impurity M by area percentage in HPLC.

Specifically, in one of the embodiment Apremilast (I) obtained in any of the inventions described herein is having less than 0.1% w/w of impurity M by area percentage in HPLC.

In fifteenth embodiment, the present application provides pharmaceutical compositions comprising apremilast of formula (I) prepared according to processes of the present application together with one or more pharmaceutically acceptable excipient, carrier and diluents.

The processes of the present invention is easy to handle, environment friendly, provides better yield with required purity and it may also be practiced at on industrial scale.

### Definitions

The following definitions are used in connection with the present invention unless the context indicates otherwise.

The term “amorphous form” as used herein refers to any amorphous solid state which is known to a person skilled in the art. For example amorphous solids lack the three-dimensional long-range order found in crystalline solids, although short-range order may be present over several molecular dimensions. Due to the lack of three-dimensional long-range order, amorphous solids do not constructively diffract X-rays, as do crystalline solids. Therefore, in X-ray powder diffraction experiments, broad, diffuse haloes are observed instead of well-defined peaks [Journal of Pharmaceutical Sciences, Vol. 93, no. 1, January 2004, Page-3].

“Substantially free of one or more of its corresponding impurities” as used herein, unless otherwise defined refers to the compound that contains less than about 2%, or less than about 1%, or less than about 0.5%, or less than about 0.3%, or less than about 0.2%, or less than about 0.1%, or less than about 0.05%, or less than about 0.03%, or less than about 0.01%, by weight, of individual impurity.

Certain specific aspects and embodiments of the present invention will be explained in more detail with reference to the following examples, which are provided for purposes of illustration only and should not be construed as limiting the scope of the present invention in any manner.

### EXAMPLES

### Example 1: Preparation of 1-(3-ethoxy-4-methoxyphenyl)-2-(methylsulfonyl) ethen-1-amine (IV)

Dimethyl sulfone (53.1 g, 564 mmoles) was added to mixture of tetrahydrofuran (1000 ml) and dimethylsulfoxide (50 ml) and stirred at room temperature for 5-10 minutes. Sodium amide (22.02 g, 564 mmoles) was added slowly to the reaction mixture at 40° C. and stirred for 10-15 minutes. 3-ethoxy-4-methoxy benzonitrile (50 g, 282 mmoles) in tetrahydrofuran (200 ml) was added to the reaction mixture over a period of 30 minutes and was stirred for three hours at room temperature. The reaction mixture was distilled under reduced pressure at 40-45° C. Water (750 ml) was added to the reaction mass and stirred at 35-40° C. for 30 minutes. The solid was filtered, washed with water (150 ml) and dried under reduced pressure at 45-50° C. to give the title compound.

Yield: 63.7 g (83%)

### Example 2: Preparation of 1-(3-Ethoxy-4-methoxyphenyl)-2-methanesulfonyl-ethylamine (II)

1-(3-ethoxy-4-methoxyphenyl)-2-(methylsulfonyl)ethen-1-amine (IV) (5 g, 18.43 mmoles) was added to methanol (25 ml) and cooled to 5° C. Methanolic hydrochloric acid (10%, 5 ml) was added to reaction mixture. Sodium cyano borohydride (1.7 g, 63.85 mmoles) was added to the reaction mixture and maintained at 5° C. for three hours. Methanol was distilled at 35° C. Water (5 ml) was added to the reaction mixture at room temperature and pH adjusted to 12-13 with 10% sodium hydroxide solution (8 ml) at 5° C. The reaction mixture was stirred at 5° C. for three hours. The solid was filtered, washed with water (5 ml) and dried under reduced pressure at 50-55° C. to give the title compound.

Yield: 2.7 g (53.68%)

### Example 3: Preparation of 1-(3-Ethoxy-4-methoxyphenyl)-2-methanesulfonyl-ethylamine II)

1-(3-ethoxy-4-methoxyphenyl)-2-(methylsulfonyl)ethen-1-amine (IV) (5 g, 18.43 mmoles) was added to methanol (125 ml) at room temperature. Citric acid (7 g, 36.4 mmoles) was added to reaction mixture. Sodium cyano borohydride (1.7 g, 63.85 mmoles) was added to the reaction mixture at 5° C. and maintained for one hour. Methanol was distilled at 35° C. Water (50 ml) was added to the reaction mixture at room temperature and pH adjusted to 12-13 with 20% sodium hydroxide solution (15 ml) at 5° C. The reaction mixture was stirred at 5° C. for three hours. The solid was filtered, washed with water (15 ml) and dried under reduced pressure at 50-55° C. to give the title compound.

Yield: 4.4 g (88%)

### Example 4: Preparation of 1-(3-Ethoxy-4-methoxy-phenyl)-2-methanesulfonyl-ethylamine (II)

Dimethyl sulfone (0.639 g, 0.067 mmoles) was added to dimethylsulfoxide (10 ml) and stirred at room temperature for 5-10 minutes. Potassium tertiary butoxide (1.89 g, 0.169 mmoles) was added slowly to the reaction mixture at 30° C. and stirred for three hours. 3-ethoxy-4-methoxy benzonitrile (1.0 g, 0.056 mmoles) in tetrahydrofuran (2 ml) was added to the reaction mixture over a period of 30 minutes and was stirred for three hours at room temperature. Sodium borohydride (0.213 g, 0.056 mmoles) was added to the reaction mixture at 30° C. and maintained for one hour. Ammonium chloride (10 ml) was added to the reaction mixture and extracted with ethyl acetate (20 ml). The ethyl acetate layer was washed with water (10 ml). The ethyl acetate layer was distilled at 30° C. to provide crude compound. The crude compound was washed with methyl tertiary butyl ether to provide the title compound as pale yellow colored solid.

Yield: 750 mg (50%)

### Example 5: Resolution of 1-(3-Ethoxy-4-methoxy-phenyl)-2-methanesulfonyl-ethylamine (II) using (−)-dibenzoyl-L-tartaric acid

1-(3-Ethoxy-4-methoxy-phenyl)-2-methanesulfonyl-ethylamine (II) (95 g, 348 mmoles) and (2R,3R)-2,3-bis(benzoyloxy)succinic acid (91 g, 243 mmoles) were added to water (1805 ml) and acetic acid (332.5 ml) and stirred at room temperature. The temperature of reaction was slowly raised to 97° C. and maintained for three hours. The reaction mass was cooled to room temperature and solid was separated. The solid was filtered and was washed with water (100 ml). The solid was dried at 70° C. for ten hours to give (−)-dibenzoyl-L-tartaric acid salt of 1-(3-ethoxy-4-methoxyphenyl)-2-(methylsulfonyl)ethylamine.

Yield: 78.3 g (35.6%);

Chiral purity: 98.10%

### Example 6: Resolution of 1-(3-Ethoxy-4-methoxy-phenyl)-2-methanesulfonyl-ethylamine (II) using (2R,3R)-2,3-bis((4-methylbenzoyl)oxy)succinic Acid

1-(3-Ethoxy-4-methoxy-phenyl)-2-methanesulfonyl-ethylamine (II) (20 g, 73.2 mmoles) and (2R,3R)-2,3-bis(benzoyloxy)succinic acid monohydrate (28.3 g, 73.2 mmoles) were added to dimethylformamide (20 ml) and methanol (200 ml) and stirred at room temperature. The temperature of reaction was slowly raised to 65° C. and maintained for two hours. The reaction mass was cooled to room temperature. The solid separated was filtered under vacuum at 30° C. and was washed with methanol (10 ml). The solid was taken in methanol (200 ml) and dimetylformamide (40) and heated to 65° C. The reaction mixture was maintained at 65° C. for two hours and allowed to cool to room temperature. The solid was filtered under vacuum at 30° C., washed with methanol (10 ml) and dried at 70° C. for two hours. The solid was taken in methanol (168 ml) and dimethylformamide (72 ml) and heated to 65° C. The reaction mixture was maintained at 65° C. for two hours and allowed to cool to room temperature. The solid was filtered under vacuum at 30° C., washed with methanol (10 ml) and dried at 70° C. for two hours to give of (2R,3R)-2,3-bis((4-methylbenzoyl)oxy)succinic acid salt of 1-(3-ethoxy-4-methoxyphenyl)-2-(methylsulfonyl) ethylamine.

Yield: 48.3 g (12.4%);

Chiral purity: 91.02%

### Example 7: Preparation of (S)-1-(3-Ethoxy-4-methoxy-phenyl)-2-methanesulfonyl-ethylamine of the Formula (Va)

(−)-Dibenzoyl-L-tartaric acid salt of 1-(3-ethoxy-4-methoxyphenyl)-2-(methylsulfonyl) ethylamine (15 g, 0.079 moles) was added to water (150 ml) and stirred at room temperature. 10% aqueous sodium hydroxide solution (25 ml) was added to the reaction mixture and stirred at room temperature. Toluene (150 ml) was added to the reaction mixture and stirred for three hours at room temperature. The layers were separated and dichloromethane (75 ml) was added to the aqueous layer and stirred for 30 minutes at room temperature. The organic layer was separated and distilled under vacuum at below 50° C. to provide the title compound as product.

Yield: 6.5 g

### Example 8: Preparation of (S)-1-(3-Ethoxy-4-methoxy-phenyl)-2-methanesulfonyl-ethylamine of the Formula (Va)

Sodium hydroxide (7 g, 0.175 moles) was added to water (110 ml) at room temperature and cooled to 0-5° C. (−)-Dibenzoyl-L-tartaric acid salt of 1-(3-ethoxy-4-methoxyphenyl)-2-(methylsulfonyl)ethylamine (50 g, 0.079 moles) was added to reaction mixture and stirred at 0-5° C. for two hours. The reaction mixture was filtered and wet solid was washed with water (50 ml). The solid was dried in oven at 60-75° C. to provide the title compound.

Yield: 18.5 g

### Example 9: Preparation of 1-(3-ethoxy-4-methoxyphenyl)-2-(methylsulfonyl)ethen-1-amine (IV)

(R)-1-(3-ethoxy-4-methoxy-phenyl)-2-methanesulfonyl-ethylamine of formula (Vb) (1.40 g, 5.12 mmol, 82.9% ee) was dissolved in dichloromethane (60 mL) at room temperature. Water (15 mL) was added to the reaction mixture and the pH of the solution was 7.6. NaDCC (707 mg, 2.76 mmol) was added to reaction mixture at 25° C. and the pH of the mixture increased to 8.5. The mixture was vigorously stirred for 45 minutes at 25° C. The organic layer was washed with water (30 mL) and the aqueous phase was separated. DBU (825 μL, 5.52 mmol) was added to the organic layer and it was stirred for 1 h at room temperature. The reaction mixture was washed with water (10 mL) and brine (10 mL), dried over magnesium sulfate and evaporated to dryness to provide the title compound as a yellow solid.

Yield: 1.38 g (99%).

### Example 10: Preparation of 1-(3-ethoxy-4-methoxyphenyl)-2-(methylsulfonyl)ethen-1-amine (IV)

(R)-1-(3-ethoxy-4-methoxy-phenyl)-2-methanesulfonyl-ethylamine of formula (Vb) containing 20% N-Ac-Leucine (1.56 g, 5.06 mmol, 82.9% ee) was dissolved in dichloromethane (60 mL) at room temperature. Water (15 mL) was added and the pH of the solution was adjusted to 9.5 with 1M sodium hydroxide. NaDCC (712 mg, 2.76 mmol) was added to the reaction mixture and the pH decreased to 8.9. The mixture was vigorously stirred for 45 minutes at 25° C. Water (30 mL) was added to the reaction mixture and layers were separated. DBU (825 μL, 5.52 mmol) was added to the organic layer and stirred for 1 h at room temperature. The reaction mixture was washed with water (10 mL) and brine (10 mL), dried over magnesium sulfate and evaporated to dryness to provide the title compound as a yellow solid.

Yield: 1.40 g (>99%).

### Example 11: 1-(3-Ethoxy-4-methoxy-phenyl)-2-methanesulfonyl-ethylamine (II)

Enamine of formula (IV) (503 mg, 1.85 mmol) was dissolved in a mixture of tetrahydrofuran (3 mL) and methanol (10 mL). Citric acid (889 mg, 4.62 mmol) was added to the reaction mixture and stirred at 30° C. for 15 min and then, cooled to 0° C. Sodium borohydride (142 mg, 3.75 mmol) was added to reaction mixture portion wise at 0° C., keeping the temperature below 5° C. The mixture turned foggy towards the end of the addition and was stirred for 1 h at 0° C. Water (10 mL) was added to the reaction mixture and the aqueous layer was washed with ethyl acetate (20 mL). The aqueous solution having pH 3-4 was separated, cooled to 0° C. and basified to pH 11-12 using 5M potassium hydroxide. A white solid precipitated upon stirring for 30 min at 0° C. The white solid was filtered off, washed with water (15 mL) and dried under vacuum to give the title compound.

Yield: 255 mg (50%).

### Example 12: Preparation of Apremilast (I)

N-(1,3-dioxo-1,3-dihydroisobenzofuran-4-yl)acetamide (1.705 g, 8.31 mmoles) and (−)-dibenzoyl-L-tartaric acid salt of 1-(3-ethoxy-4-methoxyphenyl)-2-(methylsulfonyl)ethylamine (5 g, 7.92 mmoles) were added to acetic acid (50 ml) and stirred at room temperature. The temperature of reaction was slowly raised to 95° C. and maintained for fifteen hours. The reaction mixture was distilled under reduced pressure at 60-65° C. for thirty minutes. Ethyl acetate (50 ml) and saturated bi carbonate solution (30 ml) was added to the reaction mass and stirred at 35-40° C. for two hours. The solid was filtered under vacuum at 30° C. and was washed with water (5 ml). Ethanol (30 ml) and acetone (15 ml) was added to the solid and heated to 55° C. and maintained for 2 hours. The solid was filtered under vacuum at 30° C. and dried at 65° C. for five hours to give apremilast as a product.

### Example 13: Preparation of Apremilast (I)

N-(1,3-dioxo-1,3-dihydroisobenzofuran-4-yl)acetamide (5 g, 7.92 mmoles) and (−)-dibenzoyl-L-tartaric acid salt of 1-(3-ethoxy-4-methoxyphenyl)-2-(methylsulfonyl)ethylamine (1.70 g, 8.31 mmoles) were added to dimethylformamide (25 ml) and stirred at room temperature. The temperature of reaction was slowly raised to 95° C. and maintained for eight hours. Water (50 ml) and ethyl acetate (20 ml) was added to the reaction mass at room temperature and stirred for fifteen minutes. The solid was filtered and slurried in hexane (20 ml). The solid was filtered and dried at 65° C. for thirty minutes to give apremilast as a product.

Yield: 2.8 g (77%)

### Example 14: Preparation of Apremilast (I)

(S)-1-(3-ethoxy-4-methoxyphenyl)-2-(methylsulfonyl)ethylamine (6.5 g, 0.023 moles) were added to toluene (120 ml) and stirred at room temperature. Acetic acid (30 ml) and N-acetylphthalic anhydride (5.12 g, 0.024 moles) was added to the reaction mixture at room temperature. The temperature of reaction mixture was raised to 100-105° C. and maintained for 8-10 hours. Distilled the solvent at below 60° C. under vacuum and cooled the reaction mixture to room temperature. Methyl ethyl ketone (150 ml) was added to the reaction mixture at room temperature. The temperature of reaction mixture was raised to 50-60° C. and 10% sodium bicarbonate solution (150 ml) was added followed by water (50 ml). The layers were separated and the organic layer was distilled upto one-fourth of its initial volume under vacuum and cooled to 0-5° C. The reaction mixture was maintained at 0-5° C. for 1-2 hours. The solid was filtered, washed with methyl ethyl ketone (15 ml) and dried in oven to provide apremilast as product.

Yield: 8.5 g

### Example 15: Preparation of Apremilast (I)

(S)-1-(3-ethoxy-4-methoxyphenyl)-2-(methylsulfonyl)ethylamine (5 g, 0.018 moles) were added to methyl ethyl ketone (50 ml) and stirred at room temperature. The reaction mixture was refluxed to remove the water under azeotropic conditions. Acetic acid (10 ml) and N-acetylphthalic anhydride (3.94 g, 0.019 moles) was added to reaction mixture at room temperature. The temperature of reaction mixture was raised to 80-85° C. and maintained for 8-10 hours. The reaction mixture was cooled to 60° C. and 10% sodium bicarbonate solution (150 ml) was added to reaction mixture at 50-60° C. The layers were separated and the organic layer was distilled under vacuum to a minimum volume and cooled the mass to 0-5° C. The reaction mixture was maintained at 0-5° C. for two hours. The solid was filtered, washed with methyl ethyl ketone (5 ml) and dried in oven to provide apremilast as product.

Yield: 6 g

### Example 16: Preparation of Apremilast (I)

(S)-1-(3-ethoxy-4-methoxyphenyl)-2-(methylsulfonyl)ethylamine (4 g, 0.014 moles) were added to methyl ethyl ketone (40 ml) and stirred at room temperature. Acetic acid (8 ml) and N-acetylphthalic anhydride (3.15 g, 0.015 moles) was added to reaction mixture at room temperature. The temperature of reaction mixture was raised to 100-105° C. and maintained for 6-10 hours. Water (40 ml) was added to the reaction mixture at 25-30° C. and the reaction mixture was stirred at 50-60° C. for fifteen minutes. The layers were separated and 10% sodium bicarbonate solution (32 ml) was added to the organic layer at 50-60° C. The layers were separated and 10% sodium chloride solution (32 ml) was added to organic layer. The organic layer was separated, distilled under vacuum to minimum volume and cooled the mass to 0-5° C. The reaction mixture was maintained at 0-5° C. for two hours. The solid was filtered, washed with methyl ethyl ketone (8 ml) and dried in oven to provide apremilast as product.

Yield: 4.8 g

### Example 17: Preparation of Apremilast (I)

Acetic acid (20 ml) was added to a mixture of (S)-1-(3-ethoxy-4-methoxyphenyl)-2-(methylsulfonyl)ethylamine (10 g, 36.6 moles) in methyl isobutyl ketone (60 ml) and stirred at room temperature. N-(1,3-dioxo-1,3-dihydroisobenzofuran-4-yl)acetamide (7.88 g, 38.4 moles) was added to the reaction mixture and heated to 96° C. and maintained for 30 hours. Water (80 ml) was added to the reaction mixture at room temperature. The organic layers were separated and washed with water (40 ml) and 10% sodium bicarbonate solution (40 ml) and dried over sodium sulfate. The organic layer was heated to 95° C. and maintained for half an hour. The reaction mixture was heated to 55° C. and seed crystal of apremilast was added. The reaction mixture was maintained at 45° C. for eight hours followed by at 25° C. for five hours under stirring. The solid was filtered, washed with methyl isobutyl ketone (20 ml) and dried under vacuum at 62° C. for twelve hours to provide the title compound as product. Yield: 12.6 g

### Example 18: Preparation of Apremilast (I)

Dimethylsulfoxide (2.5 ml) was added to a mixture of (S)-1-(3-ethoxy-4-methoxyphenyl)-2-(methylsulfonyl)ethylamine (5 g, 18.29 moles) in methyl isobutyl ketone (50 ml) and stirred at room temperature. N-(1,3-dioxo-1,3-dihydroisobenzofuran-4-yl)acetamide (3.94 g, 19.21 moles) was added to the reaction mixture and heated to 96° C. and maintained for 15 hours. Water (50 ml) was added to the reaction mixture at room temperature. The organic layers were separated and washed with water (20 ml) and 10% sodium bicarbonate solution (20 ml) and dried over sodium sulfate. The reaction mixture was stirred to 25° C. and seed crystal of apremilast was added. The reaction mixture was stirred and maintained at 25° C. for six hours. The solid was filtered, washed with methyl isobutyl ketone (10 ml) and dried under vacuum at 62° C. for four hours to provide the title compound as product.

Yield: 6.3 g

### Example 19: Preparation of Crystalline Form B of Apremilast

Apremilast (100 g, 0.217 moles) was added to methyl isobutyl ketone (900 ml) and stirred at 30-35° C. The reaction mixture was heated to 90-95° C. and stirred for 1-2 hours. The reaction mass was cooled to 75° C. and filtered through micron filter and washed with methyl isobutyl ketone (100 ml). The combined filtrate was heated to 90-95° C. The reaction mass was cooled to 40-45° C. and seed crystals of form B of apremilast were added. The reaction mass was maintained at 40-45° C. for 6-7 hours and further at 30-35° C. for 14-16 hours. The reaction mass was filtered over buchner funnel at 30-35° C. and washed with methyl isobutyl ketone (50 ml). The solid obtained was dried under vacuum at 75-85° C. for 8-9 hours to provide the title compound.

Yield: 30 g

### Example 20: Preparation of Amorphous Apremilast

Apremilast (5 g) was dissolved in methanol (150 mL) at 45° C. The clear solution was added dropwise to water (250 mL) at 0-5° C. The solid was filtered, washed with water (200 ml) and was dried at 40° C. under vacuum for about 6-8 hours to provide the title compound.

### Example 21: Preparation of Amorphous Apremilast

Apremilast (5 g) was dissolved in dimethyl sulfoxide (25 mL) at 28° C. The clear solution was added dropwise to water (80 mL) at 0-5° C. The solid was filtered, washed with water (20 ml) and was dried at 40° C. under vacuum for about 6-8 hours to provide the title compound.

### Example 22: Preparation of Amorphous Apremilast

Apremilast (5 g) was dissolved in acetonitrile (25 mL) at 50° C. The clear solution was added dropwise to water (50 mL) at 0-5° C. The solid was filtered and was dried at 25-30° C. under vacuum for about 5-6 hours to provide the title compound.

### Example 23: Preparation of Amorphous Apremilast

Apremilast (5 g) was dissolved in a mixture of methanol (8 ml) and dimethylsulfoxide (10 mL) at 28° C. The clear solution was added dropwise to water (100 mL) at 0-5° C. The solid was filtered and was dried at 25-30° C. under vacuum for about 12 hours to provide the title compound.

### Example 24: Preparation of Amorphous Apremilast

Apremilast (1 g) was dissolved in acetone (15 mL) at 50° C. The clear solution was added dropwise to water (50 mL) at 0-5° C. The solid was filtered and was dried at 25-30° C. under vacuum for about 5-6 hours to provide the title compound.

### Example 25: Preparation of Amorphous Apremilast

Apremilast (1 g) was dissolved in acetone (15 mL) at 50° C. Pre-cooled water (50 mL) was added to apremilast mixture at 0-5° C. The solid was filtered and was dried at 25-30° C. under vacuum for about 5-6 hours to provide the title compound.

### Example 26: Humidification of Amorphous Apremilast

Amorphous apremilast (1 g) was kept in a humidification chamber at 30° C. and 60% relative humidity for twenty hours and found that the amorphous form is retained as such by X-ray diffractogram.

### Example 27: Humidification of Amorphous Apremilast

Amorphous apremilast (1 g) was kept in a humidification chamber at 30° C. and 90% relative humidity for twenty hours and found that the amorphous form is retained as such by X-ray diffractogram.

### Example 28: Process for the Preparation of Desoxo Impurity of Apremilast (Impurity M)

Racemic amino sulfone of formula (II) (150 g) obtained after distillation the solvent from mother liquor from one of the plant batches, N-(1,3-dioxo-1,3-dihydroisobenzofuran-4-yl)acetamide (113 g), methyl isobutyl ketone (300 mL) and acetic acid (700 mL) were charged into a round bottom flask and stirred at room temperature. The mixture was heated to 98-100° C. and maintained for 3 hours. The solvent from the reaction mass was evaporated under vacuum at below 60° C. Dichloromethane (300 mL) water (200 mL) were charged to the reaction mass at room temperature and stirred for 10 minutes. The organic layers were separated and washed with water (2×200 mL). The combined organic layer was distilled under vacuum. Methyl isobutyl ketone (750 mL) was added to the residue and maintained for 15 hours at room temperature. The solid was filtered and washed with methyl isobutyl ketone (100 mL) and dried under vacuum at 85° C. for 10-12 hours. Dried compound was adsorbed on silica gel and subjected to flash column chromatography and impurity was separated by collecting the required fraction.

1H NMR (400 MHz, CDCl3) δ (ppm): 1.45 (s, 6H), 9.4 (2NH), 8.75 (d, 2H), 7.65 (t, 2H) 7.48 (d, 2H), 5.85 (m, 2H), 4.6 & 3.65 (m, 4H), 4.1 (4H), 7.15 (d, 4H), 6.8 (d, 2H), 3.8 (6H), 3.15 (t, 4H), 2.4 (m, 2H), 2.25 (s, 6H);

Mass Spectral data: m/Z=931 (M-1) and m/Z=955 (Na adduct).

We claim:

1) A process for preparation of aminosulfone of formula (II) or its stereo isomers and their pharmaceutically acceptable salts 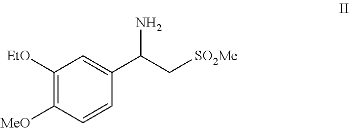 [SMILES: CCOc1cc(C(N)CS(C)(=O)=O)ccc1OC]
which comprises:
(a) reacting benzonitrile of formula (III) with dimethyl sulfone in the presence of a base in a suitable solvent to provide a compound of formula (IVa), followed by its conversion to provide enamine of formula (IV); 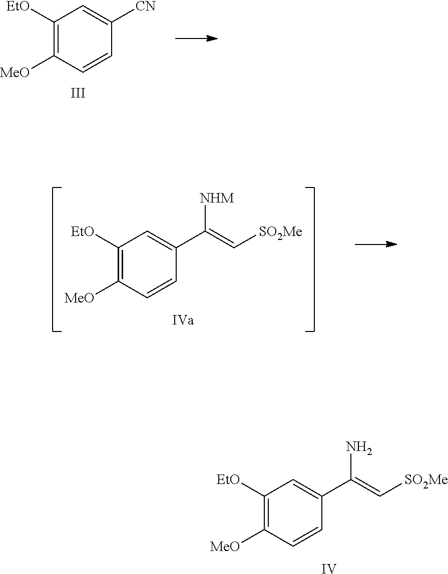
Wherein M=Na, K
(b) reducing enamine of formula (IV) in presence of a suitable solvent to provide aminosulfone of formula (II); 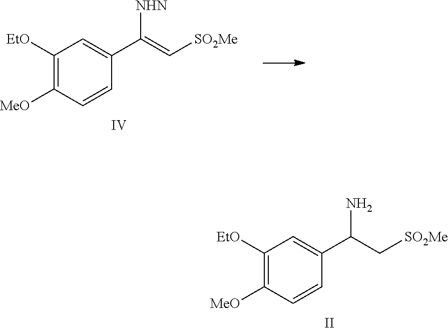
(c) optionally purifying amino sulfone of formula (II).

2) The process as claimed in claim 1, wherein base used in step a) is selected from sodium amide, potassium amide, C₁-C₂₀ alkoxide of sodium, C₁-C₂₀ alkoxide of potassium, C₁-C₂₀ alkoxide of magnesium, sodium hydride and potassium hydride.

3) The process as claimed in claim 1, wherein step b) is carried out in the presence of an acid selected from acetic acid, methanesulfonic acid, trifluoroacetic acid, 4-(trifluoromethyl)benzoic acid, p-toluenesulfonic acid, hydrochloric acid, nitric acid, sulfuric acid, phosphoric acid, citric acid, tartaric acid and benzene sulfonic acid.

4) A process for the preparation of formula (Va) or its stereoisomers thereof 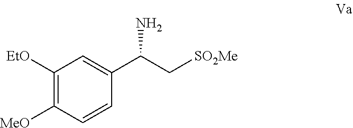 [SMILES: CCOc1cc([C@H](N)CS(C)(=O)=O)ccc1OC]
which comprises:
(a) contacting racemic 1-(3-ethoxy-4-methoxy-phenyl)-2-methanesulfonyl-ethylamine of formula (II) with a chiral acid in presence of a suitable solvent to form a chiral acid salt of 1-(3-ethoxy-4-methoxy-phenyl)-2-methanesulfonyl-ethylamine of formula (V); 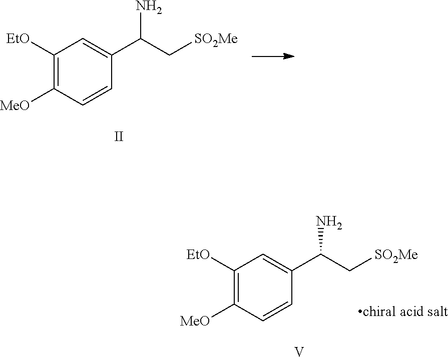
(b) optionally isolating and purifying chiral acid salt of aminosulfone of formula (V);
(c) treating chiral acid salt of aminosulfone of formula (V) with base in suitable solvent to form chiral aminosulfone of formula (Va); 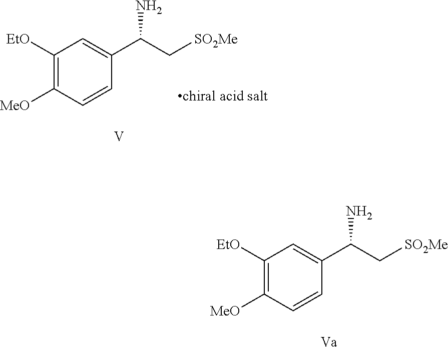
(d) optionally isolating and purifying chiral aminosulfone of formula (Va).

5) The process as claimed in claim 4, wherein chiral acid used in step a) is selected from individual enantiomers of 10-camphorsulfonic acid, camphoric acid, methoxyacetic acid, tartaric acid, diacetyltartaric acid, di-toluoyl tartaric acid, dibenzoyl tartaric acid, mandelic acid, derivatives of mandelic acid such as acetyl mandelic acid, propyl mandelic acid, lactic acid, ibuprofen, malic acid, pyrrolidone-5-carboxylic acid and naproxen.

6) The process as claimed in claim 4, wherein base used in step c) is selected from pyridine, piperidine, pyrimidine, triethylamine, diethylamine, diisopropyl ethylamine, 1,1,3,3-tetramethylguanidine, DBU, DABCO, sodium carbonate, potassium carbonate; metal bicarbonates such as sodium bicarbonate, potassium bicarbonate; metal hydroxide like sodium hydroxide, potassium hydroxide, lithium hydroxide and calcium hydroxide.

7) A process for preparation of apremilast of formula (I) or its stereoisomers thereof 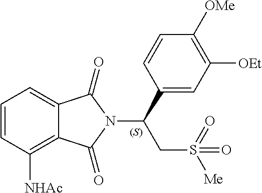
which comprises:
(a) contacting 1-(3-Ethoxy-4-methoxy-phenyl)-2-methanesulfonyl-ethylamine of formula (II) with a chiral acid in presence of a suitable solvent to form a chiral acid salt of 1-(3-ethoxy-4-methoxy-phenyl)-2-methanesulfonyl-ethylamine of formula (V); 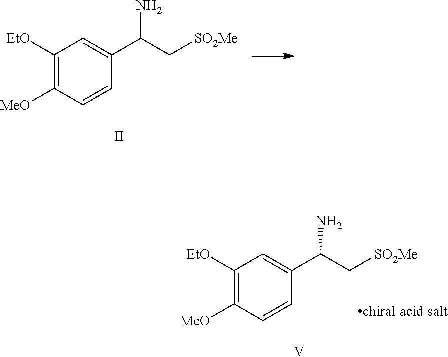
(b) optionally isolating and purifying chiral acid salt of aminosulfone of formula (V);
(c) optionally treating chiral acid salt of aminosulfone of formula (V) with base to form chiral aminosulfone of formula (Va); 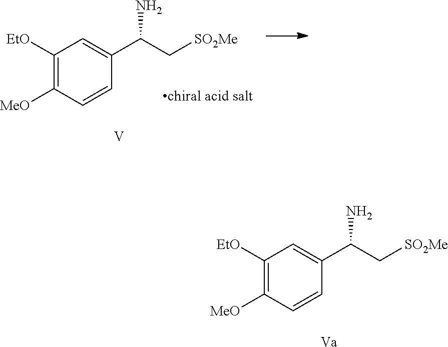
(d) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of a suitable solvent to provide apremilast of formula (I); 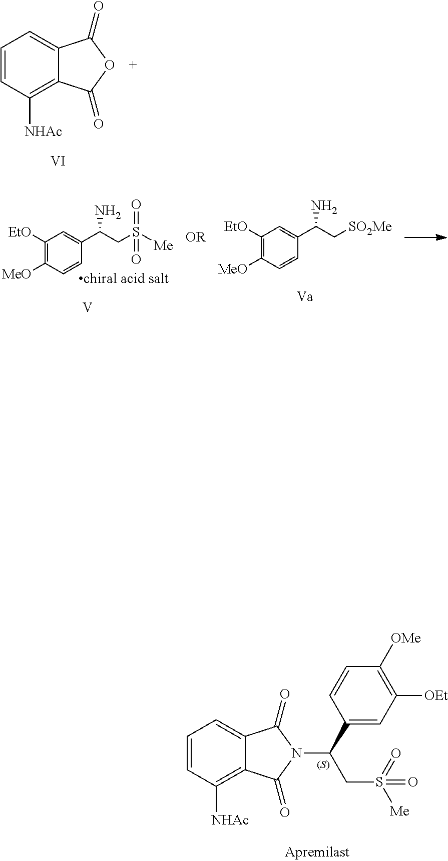
(e) optionally purifying apremilast of formula (I).

8) The process as claimed in claim 7, wherein solvent used in step d) is selected from ethers, ketone solvents, aromatic hydrocarbon solvents, nitrile solvents, alcohol solvents, ester solvents, amide solvents, acid solvents, water and mixtures thereof.

9) A process for preparation of apremilast of formula (I) or its stereoisomers thereof: 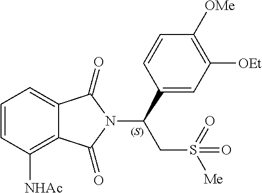
which comprises:
(a) reacting benzonitrile of formula (III) with dimethyl sulfone in the presence of a base in a suitable solvent to provide a compound of formula (IVa), followed by its conversion to provide enamine of formula (IV); 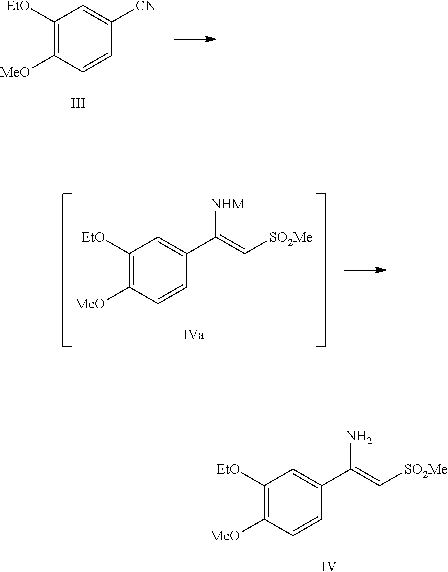
Wherein M=Na, K
(b) reducing enamine of formula (IV) in presence of a suitable solvent to provide aminosulfone of formula (II);
(c) optionally purifying amino sulfone of formula (II);
(d) contacting 1-(3-Ethoxy-4-methoxy-phenyl)-2-methanesulfonyl-ethylamine of formula (II) with a chiral acid in presence of a suitable solvent to form a chiral acid salt of 1-(3-Ethoxy-4-methoxy-phenyl)-2-methanesulfonyl-ethylamine of formula (V);
(e) optionally isolating and purifying chiral acid salt of aminosulfone of formula (V);
(f) optionally treating chiral acid salt of aminosulfone of formula (V) with base to form chiral aminosulfone of formula (Va);
(g) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of a suitable solvent to provide apremilast of formula (I);
(h) optionally purifying apremilast of formula (I).

10) A crystalline form of chiral aminosulfone of formula (Va) characterized by its powder X-ray diffraction (PXRD) pattern having peaks at about 5.97, 17.81, 19.85 and 26.07±0.2 degrees 2θ.

11) The crystalline form of chiral aminosulfone of formula (Va) according to claim 10, further comprising 2-theta peaks, located at about 11.88, 15.88, 21.96 and 26.72±0.2.

12) The crystalline form of chiral aminosulfone of formula (Va) according to claims 10 and 11, further comprising 2-theta peaks, located at about 12.10, 20.72 and 22.18±0.2.

13) A process for the preparation of racemic aminosulfone of formula (II) and its pharmaceutically acceptable salts 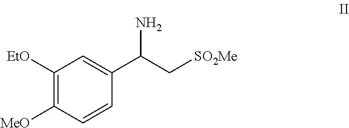 [SMILES: CCOc1cc(C(N)CS(C)(=O)=O)ccc1OC]
which comprises:
(a) reacting of (R)-1-(3-ethoxy-4-methoxy-phenyl)-2-methanesulfonyl-ethylamine of formula (Vb) and its pharmaceutically acceptable salts with a halogenating reagent in the presence of a suitable solvent to provide a halogenated amine of formula (VII); 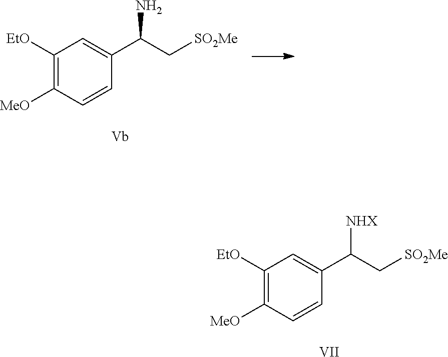
wherein X=Cl, F, Br, I
(b) treating halogenated amine of formula (VII) in presence of a suitable base in a suitable solvent to provide enamine of formula (IV); 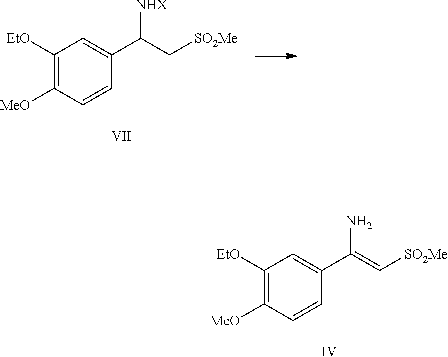
wherein X=Cl, F, Br, I
(c) converting the enamine of formula (IV) to racemic aminosulfone of formula (II) in presence of reducing agent and a suitable solvent;
(d) optionally purifying racemic amino sulfone of formula (II).

14) The process as claimed in claim 13, wherein halogenating reagent used in step a) is selected from Sodium dichloroisocyanurate (NaDCC), trichloroisocyanuric acid, N,N′-dichlorobis(2,4,6-trichlorophenyl)urea, N-chlorosuccinimide, N-bromosuccinimide, sodium hypochlorite and sodium hypobromite.

15) The process as claimed in claim 13, wherein reducing agent used in step c) is selected from sodium borohydride, lithium borohydride, sodium cyanoborohydride and di-isobutyl aluminum hydride.

16) A novel compound of formula (VII). 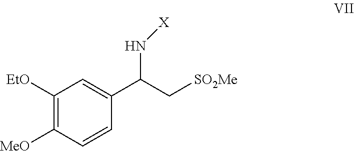
wherein X=Cl, F, Br, I

17) A process for preparation of apremilast of formula (I) or its stereoisomers thereof which comprises:
(a) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of mixture of ketonic solvent and polar solvent to provide apremilast of formula (I); 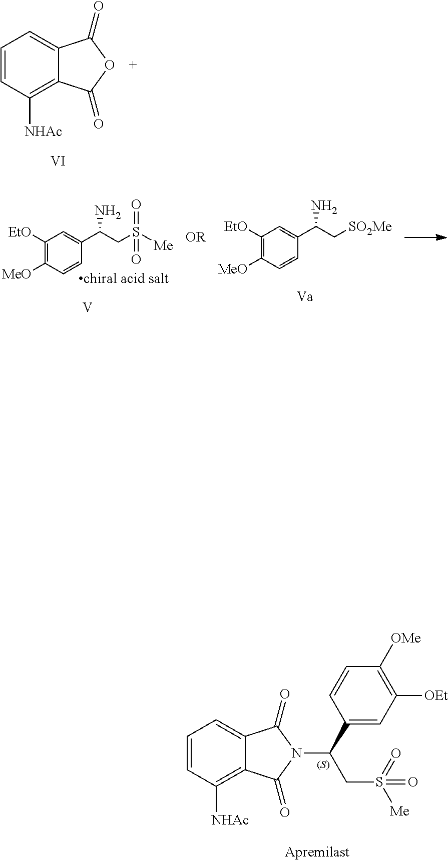
(b) optionally purifying apremilast of formula (I).

18) The process as claimed in claim 17, wherein ketonic solvent used in step a) is selected from acetone, dialkyl ketone, ethyl methyl ketone, methyl isobutyl ketone and mixtures thereof.

19) The process as claimed in claim 17, wherein polar solvent used in step a) is selected from acid solvents, ethers, nitriles, esters, alcohols, amides, water and mixtures thereof.

20) A process for preparation of crystalline form B of apremilast of formula (I) or its stereoisomers thereof: which comprises:
(a) contacting the chiral acid salt of 1-(3-Ethoxy-4-methoxy-phenyl)-2-methanesulfonyl-ethylamine of formula (V) or chiral aminosulfone of formula (Va) with N-(1,3-Dioxo-1,3-dihydro-isobenzofuran-4-yl)-acetamide of formula (VI) in presence of mixture of ketonic solvent and polar solvent to provide apremilast of formula (I); 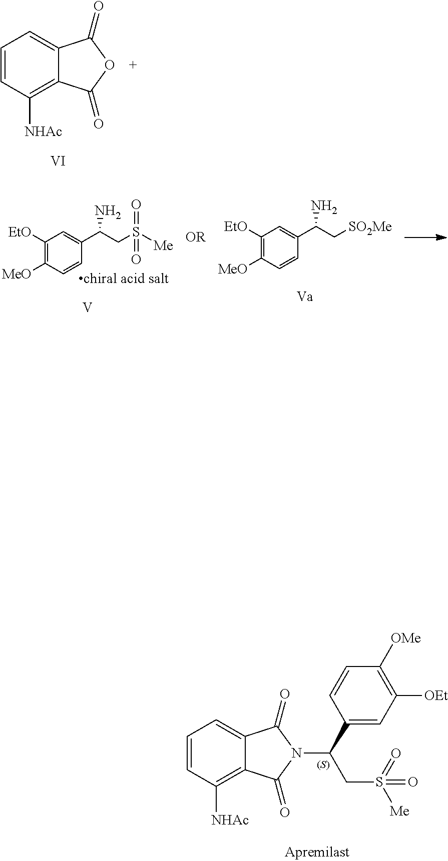
(b) optionally isolating and purifying apremilast;
(c) converting the apremilast obtained in step (b) to crystalline form B of apremilast;
(d) optionally isolating and purifying crystalline form B of apremilast.

21) A process for preparing amorphous form of apremilast comprising:
a) dissolving apremilast in a suitable solvent or mixture thereof;
b) optionally, heating the solution of step (a);
c) adding water as an anti-solvent to the solution of apremilast; or adding solution of apremilast to water;
d) isolating the solid;
e) optionally, drying the product at suitable temperature.

22) The process as claimed in claim 21, wherein solvent used in step a) is selected from dimethylformamide; dimethylacetamide; dimethyl sulphoxide, nitriles, ketones, ethers, esters, halogenated hydrocarbons, alcohols and mixtures thereof.

23) A compound of desoxo impurity of Apremilast (Impurity M).

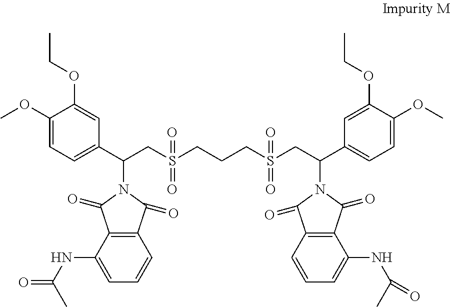

24) Apremilast of formula (I) substantially free of desoxo impurity (Impurity M).
